SEQUENCE LISTING (64 sequences)

SEQ ID NO: 1 - DNA, 20 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
nnnvtnnnvtnnnvtnnnvt

SEQ ID NO: 2 - DNA, 20 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
nnnbannnbannnbannnba

SEQ ID NO: 3 - DNA, 20 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
nnnhgnnnhgnnnhgnnnhg

SEQ ID NO: 4 - DNA, 20 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
nnndcnnndcnnndcnnndc

SEQ ID NO: 5 - DNA, 20 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
tvnnntvnnntvnnntvnnn

SEQ ID NO: 6 - DNA, 20 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
abnnnabnnnabnnnabnnn

SEQ ID NO: 7 - DNA, 20 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
ghnnnghnnnghnnnghnnn

SEQ ID NO: 8 - DNA, 20 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
cdnnncdnnncdnnncdnnn

SEQ ID NO: 9 - DNA, 15 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
nnnvtnnnvtnnnvt

SEQ ID NO: 10 - DNA, 15 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
nnnbannnbannnba

SEQ ID NO: 11 - DNA, 15 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
nnnhgnnnhgnnnhg

SEQ ID NO: 12 - DNA, 15 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
nnndcnnndcnnndc

SEQ ID NO: 13 - DNA, 15 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
tvnnntvnnntvnnn

SEQ ID NO: 14 - DNA, 15 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
abnnnabnnnabnnn

SEQ ID NO: 15 - DNA, 15 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
ghnnnghnnnghnnn

SEQ ID NO: 16 - DNA, 15 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
cdnnncdnnncdnnn

SEQ ID NO: 17 - DNA, 10 bp, Artificial Sequence
  Synthetic Construct
  misc_feature (1)..(3): n is a, c, g, or t
  misc_feature (6)..(8): n is a, c, g, or t
nnnvtnnnvt

SEQ ID NO: 18 - DNA, 10 bp, Artificial Sequence
  Synthetic Construct
  misc_feature (1)..(3): n is a, c, g, or t
  misc_feature (6)..(8): n is a, c, g, or t
nnnbannnba

SEQ ID NO: 19 - DNA, 10 bp, Artificial Sequence
  Synthetic Construct
  misc_feature (1)..(3): n is a, c, g, or t
  misc_feature (6)..(8): n is a, c, g, or t
nnnhgnnnhg

SEQ ID NO: 20 - DNA, 10 bp, Artificial Sequence
  Synthetic Construct
  misc_feature (1)..(3): n is a, c, g, or t
  misc_feature (6)..(8): n is a, c, g, or t
nnndcnnndc

SEQ ID NO: 21 - DNA, 10 bp, Artificial Sequence
  Synthetic Construct
  misc_feature (3)..(5): n is a, c, g, or t
  misc_feature (8)..(10): n is a, c, g, or t
tvnnntvnnn

SEQ ID NO: 22 - DNA, 10 bp, Artificial Sequence
  Synthetic Construct
  misc_feature (3)..(5): n is a, c, g, or t
  misc_feature (8)..(10): n is a, c, g, or t
abnnnabnnn

SEQ ID NO: 23 - DNA, 10 bp, Artificial Sequence
  Synthetic Construct
  misc_feature (3)..(5): n is a, c, g, or t
  misc_feature (8)..(10): n is a, c, g, or t
ghnnnghnnn

SEQ ID NO: 24 - DNA, 9 bp, Artificial Sequence
  Synthetic Construct
  misc_feature (3)..(5): n is a, c, g, or t
  misc_feature (8)..(9): n is a, c, g, or t
cdnnncdnn

SEQ ID NO: 25 - DNA, 25 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
  misc_feature (21)..(23): n is a, c, g, or t
nnnvtnnnvtnnnvtnnnvtnnnvt

SEQ ID NO: 26 - DNA, 25 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
  misc_feature (21)..(23): n is a, c, g, or t
nnnbannnbannnbannnbannnba

SEQ ID NO: 27 - DNA, 25 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
  misc_feature (21)..(23): n is a, c, g, or t
nnnhgnnnhgnnnhgnnnhgnnnhg

SEQ ID NO: 28 - DNA, 25 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
  misc_feature (21)..(23): n is a, c, g, or t
nnndcnnndcnnndcnnndcnnndc

SEQ ID NO: 29 - DNA, 25 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
  misc_feature (23)..(25): n is a, c, g, or t
tvnnntvnnntvnnntvnnntvnnn

SEQ ID NO: 30 - DNA, 25 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
  misc_feature (23)..(25): n is a, c, g, or t
abnnnabnnnabnnnabnnnabnnn

SEQ ID NO: 31 - DNA, 25 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
  misc_feature (23)..(25): n is a, c, g, or t
ghnnnghnnnghnnnghnnnghnnn

SEQ ID NO: 32 - DNA, 25 bp, Artificial Sequence
  Synthetic Construct
  misc_feature (3)..(5): n is a, c, g, or t
  misc_feature (8)..(10): n is a, c, g, or t
  misc_feature (13)..(15): n is a, c, g, or t
  misc_feature (18)..(20): n is a, c, g, or t
  misc_feature (23)..(25): n is a, c, g, or t
cdnnncdnnncdnnncdnnncdnnn

SEQ ID NO: 33 - DNA, 20 bp, Artificial Sequence
  Synthetic Construct
  misc_feature (1)..(3): n is a, c, g, or t
  misc_feature (6)..(8): n is a, c, g, or t
  misc_feature (11)..(13): n is a, c, g, or t
  misc_feature (16)..(18): n is a, c, g, or t
nnnvtnnnvtnnnvtnnnvt

SEQ ID NO: 34 - DNA, 39 bp, Artificial Sequence
  Synthetic Construct
atttgagggcttcccacccctgcgtcccagaagttccac

SEQ ID NO: 35 - DNA, 38 bp, Artificial Sequence
  Synthetic Construct
gtgttgagggcttcccaccccgcgtccctgagttccac

SEQ ID NO: 36 - DNA, 39 bp, Artificial Sequence
  Synthetic Construct
atttgagggctttacccacctgcgtccccgaagttccgt

SEQ ID NO: 37 - DNA, 40 bp, Artificial Sequence
  Synthetic Construct
atttaaggggagcttcccaccccctgtcccagagttccac

SEQ ID NO: 38 - DNA, 32 bp, Artificial Sequence
  Synthetic Construct
gttgggggcttccacctgcgtcccagttccac

SEQ ID NO: 39 - DNA, 39 bp, Artificial Sequence
  Synthetic Construct
atttgagggcttttcccacctcgtccccagaagttccac

SEQ ID NO: 40 - DNA, 36 bp, Artificial Sequence
  Synthetic Construct
atcttgaggcttcccacccctgcgtccccagaagtt

SEQ ID NO: 41 - DNA, 37 bp, Artificial Sequence
  Synthetic Construct
gttgaggggcttcccacccctgcgtcccaagttccac

SEQ ID NO: 42 - DNA, 40 bp, Artificial Sequence
  Synthetic Construct
attgagggcttctaccccacccctgcgtcccaagttccac

SEQ ID NO: 43 - DNA, 43 bp, Artificial Sequence
  Synthetic Construct
atttgagggcttctaccccactcactgcgtcccagaagttcac

SEQ ID NO: 44 - DNA, 38 bp, Artificial Sequence
  Synthetic Construct
atttgaggcttcccacctgcgtctacaggggtttccac

SEQ ID NO: 45 - DNA, 34 bp, Artificial Sequence
  Synthetic Construct
ttgagggcacccaccccgcgctcccaagttccac

SEQ ID NO: 46 - DNA, 39 bp, Artificial Sequence
  Synthetic Construct
atttgaggggcttcccacctgcgtccccacggattccac

SEQ ID NO: 47 - DNA, 43 bp, Artificial Sequence
  Synthetic Construct
atttgaggggcttccacccccccctggcttcccagaagttcct

SEQ ID NO: 48 - DNA, 36 bp, Artificial Sequence
  Synthetic Construct
atttgagggcttcccaccccaccgtcccagttccac

SEQ ID NO: 49 - DNA, 39 bp, Artificial Sequence
  Synthetic Construct
gtgtgggagggcttcccacccctgcgtcccaagttcccc

SEQ ID NO: 50 - DNA, 31 bp, Artificial Sequence
  Synthetic Construct
atttagggctttacctacgtcaaagttccac

SEQ ID NO: 51 - DNA, 39 bp, Artificial Sequence
  Synthetic Construct
atttgagggctttcccacccccctgcgtccaagttccac

SEQ ID NO: 52 - DNA, 30 bp, Artificial Sequence
  Synthetic Construct
atttggggcttcccacctgcgtcccaagta

SEQ ID NO: 53 - DNA, 39 bp, Artificial Sequence
  Synthetic Construct
attgagggcttcccacccctaccgtcccagggcgtttac

SEQ ID NO: 54 - DNA, 37 bp, Artificial Sequence
  Synthetic Construct
attgtagggctggcccacccctgcgtccaagttccac

SEQ ID NO: 55 - DNA, 37 bp, Artificial Sequence
  Synthetic Construct
attgaggcattcccacccctgcgtccagaagttccac

SEQ ID NO: 56 - DNA, 34 bp, Artificial Sequence
  Synthetic Construct
atttgagggcttccacccctgcgttgagttccac

SEQ ID NO: 57 - DNA, 39 bp, Artificial Sequence
  Synthetic Construct
attgaggggcttcccacccccctgcgtcccagagttcct

SEQ ID NO: 58 - DNA, 40 bp, Artificial Sequence
  Synthetic Construct
atttgaggggcttcccacccctgcgtcccaggagttccac

SEQ ID NO: 59 - DNA, 44 bp, Artificial Sequence
  Synthetic Construct
atttgagggctacccaccccccccctgcgtccccagaagttcac

SEQ ID NO: 60 - DNA, 39 bp, Artificial Sequence
  Synthetic Construct
atttgggggcttcccacccctgcgtcccagaagttccac

SEQ ID NO: 61 - DNA, 42 bp, Artificial Sequence
  Synthetic Construct
attagaggctccaccacccctgcgtccccagaagttccccac

SEQ ID NO: 62 - DNA, 25 bp, Artificial Sequence
  Synthetic Construct
acccctgcgtcccagaagttcccac

SEQ ID NO: 63 - DNA, 33 bp, Artificial Sequence
  Synthetic Construct
atttgagggcttcccacccctgctcggttccac

SEQ ID NO: 64 - DNA, 40 bp, Artificial Sequence
  Synthetic Construct
atttgagggcttcccaccccctgcgtcccagaagttccac

(19) United States
(12) Patent Application Publication
(10) Pub. No.: US 2020/0208140 A1
(43) Pub. Date: Jul. 2, 2020

(54) METHODS OF MAKING AND USING TANDEM, TWIN BARCODE MOLECULES

(71) Applicant: Ohio State Innovation Foundation, Columbus, OH (US)

(72) Inventors: Sanggu KIM, Columbus, OH (US); Hannah YU, Columbus, OH (US); Alice BAEK, Columbus, OH (US)

(21) Appl. No.: 16/643,206

(22) PCT Filed: Aug. 31, 2018
(86) PCT No.: PCT/US2018/049203

§ 371 Date: Feb. 28, 2020

Related U.S. Application Data
(60) Provisional application No. 62/552,847, filed on Aug. 31, 2017.

(51) Int. Cl. C12N 15/10; C12Q 1/6874

(52) U.S. Cl. CPC C12N 15/1065; C12Q 1/6874

(57) ABSTRACT

Disclosed herein are methods related to the production of tandem, twin barcode (TTB) molecules. These TTB molecules are useful in sequencing to identify and resolve errors.

### CROSS-REFERENCE TO RELATED APPLICATIONS

This application is a national stage application filed under 35 U.S.C. § 371 of PCT/US2018/049203 filed Aug. 31, 2018, which claims benefit of U.S. Provisional Application No. 62/552,847, filed Aug. 31, 2017, incorporated herein by reference in its entirety.

### BACKGROUND

Innovations in sequencing technologies over the past decade have been critical driving forces accelerating the ongoing revolution in medicine and the life sciences and opening up new research and business opportunities with boundless potential. The growth of sequencing-based research and business opportunities is highly dependent upon the technological strength of a given sequencing platform. Although the low-cost, massively high-throughput sequencing capacities of the “next-generation” (NGS) sequencing platforms have already made major impacts on science and medicine, there is an unmet need for long read length and low error rates—this is slowing the growth of the sequencing field. The Nanopore® technology, a newer “3rd generation” single-strand real-time sequencing technology, has several revolutionary innovations, including a tremendously long read length capacity (company record: 350 Kb in a single read), real-time data output, and pocket-size mobility (thanks to the MinION sequencer). The technology, however, has disappointingly high sequence error rates (˜30% for the R7, 1D version, and 10-20% in the R9 version).

Barcodes are unique nucleic acid sequences incorporated into DNA molecules and can be used to identify the sample from which the DNA was taken. Barcoding (also known as molecular tagging) has been a powerful technique for studying the genetic and functional variations of the target pool. In particular, barcoding-mediated error correction methods have significantly improved the accuracy of sequencing individual DNA molecules with NGS platforms. Individual target DNA molecules can be accurately sequenced by barcoding the individual molecules, then amplifying and sequencing the barcoded DNA for the purpose of error correction. The utility of conventional barcoding approaches greatly depends upon the sequence read accuracy of a given sequencing platform. For example, sequencing 14-20 bp barcodes with a NGS platform will result in 2-20% read errors in all barcodes. These errors, including type I (false positive) errors and misidentification of different barcodes (collision), lead to over-estimation, cross-contamination, and erroneous quantification of the barcoded DNA, thereby significantly limiting the application of current barcoding approaches. In particular, the third-generation, Nanopore sequencing platform will have read errors in nearly all barcodes given its current error rates (an average of one error in every 20 bases). This disappointingly high error rate significantly limits the applications of barcoding approaches for Nanopore sequencing, including the barcoding-mediated sequence error-correction method which would otherwise have enabled the development of a long-range, high-accuracy sequencing platform capitalizing on the Nanopore's unique features.

What is needed in the art are more accurate, dependable sequencing tools, and methods that can eliminate barcode-reading errors and (ii) methods that use them to improve various barcoding approaches, including the barcoding-mediated high-accuracy sequencing method.

### SUMMARY

Disclosed herein is a library of tandem twin barcode (TTB) oligonucleotide molecules, wherein said library comprises at least 5 unique TTB oligonucleotide molecules, wherein said TTB molecules comprise a first and second barcode sequence, wherein said first and second barcode sequence are identical to each other and positioned in a same 5′ to 3′ orientation, and wherein said TTB oligonucleotide molecules are flanked on either side by two target regions that are common to all TTB oligonucleotides in the library.

Also disclosed herein is a method of labeling target polynucleotide molecules with a unique identifier, the method comprising labeling the barcode library with target polynucleotide molecules. For example, the target polynucleotide can be sequenced after labeling with the barcode library.

Also disclosed is a kit for labelling a target nucleic acid for sequencing, wherein the kit comprises a) a library of at least 5 unique TTB molecules, wherein said TTB molecules comprise a first and second barcode sequence, wherein said first and second barcode sequence are identical to each other; and b) reagents for sequencing. The kit can comprise various molecular biology reagents, including DNA polymerases, RNA polymerases, Reverse-transcriptases, DNA ligases, RNA ligases, transposases, viral integrase, CRISPR/Cas9, zinc finger nucleases, transcription activator-like effector nucleases, exonucleases, endonucleases, Polynucleotide Kinases, or nucleotides.

Disclosed herein is a method of making a tandem twin barcode (TTB) molecules comprising: (a) providing single, barcoded oligomers; (b) ligating single, barcoded oligomers to form a circularized, single barcoded oligomer; (c) synthesizing a complementary strand of the circularized, single barcoded oligomer to form two barcoded oligomers, where one is a sense strand and one is an antisense strand; (d) nicking 5′ upstream of both sense and antisense oligomers so that each barcode region of sense and antisense oligomers are now single-stranded; (e) synthesizing single-stranded regions of both sense and antisense oligomers to fill in barcoded regions, thereby creating a double-barcoded region on both the sense oligomer and the antisense oligomer; (f) nicking the antisense oligomer in order to differentiate sense and antisense strands oligomer, so that the antisense oligomer is shorter than the sense oligomer; (g) isolating the sense oligomer by denaturation of nicked molecules followed by separation of sense and antisense strands; and (h) circularizing single-stranded, double-barcoded oligomers, thereby forming tandem, twin barcode molecules.

Also disclosed herein is a method of sequencing individual nucleic acid molecules in a sample comprising a plurality of nucleic acid molecules, the method comprising: (a) labeling individual target nucleic acid molecules by annealing, synthesizing, inserting, or ligating tandem, twin barcode oligonucleotide molecules to the 3′ end of the target nucleic acid molecules, thereby creating a barcoded, sense-stranded nucleic acid molecule; (b) using primers specific to the bound tandem, twin barcoded nucleic acid molecules to produce amplicons with a tandem, twin barcode molecule embedded therein; (c) sequencing each amplicon with a tandem, twin barcode molecule embedded therein to produce individual sequence reads; (d) cross-comparing the tandem, twin barcode molecules within a same sequence read to correct any read errors within the barcodes; (e) grouping of nucleic acid sequence reads with identical barcodes; (f) resolving errors in sequencing by forming a consensus of correct nucleic acid sequences; and g) determining correct nucleic acid sequence for each individual nucleic acid molecule.

Disclosed herein is a method of counting nucleic acid molecules in a sample, wherein the sample comprises multiple, different nucleic acids, the method comprising: a) attaching a TTB oligonucleotide molecule to each of the plurality of nucleic acid molecules in the sample to produce a plurality of differently barcoded nucleic acid molecules, wherein said TTB molecules comprise a first and second barcode sequence, wherein said first and second barcode sequence are identical to each other and positioned in a same 5′ to 3′ orientation, and wherein said TTB oligonucleotide molecules are flanked on either side by two target regions; and b) amplifying the plurality of differently barcoded nucleic acid molecules in the sample to produce amplicons of the plurality of differently TTB barcoded nucleic acid molecules.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the preparation procedure for tandem, twin barcodes (TTB). Chemically synthesized single-barcode primers are circularized using CircLigase (Illumina) (Step 1). The second strand of the circularized DNA are then synthesized by DNA polymerase-mediated primer extension (Step 2) and treated with two nicking endonucleases (arrows) to separate the sense and anti-sense barcodes (Step 3). The second strands of barcodes are synthesized by 5′ to 3′ fill-in (Step 4). One of the two strands are isolated by strand-specific nicking (arrow), followed by the purification of the intact ssDNA (Step 5); the isolated ssDNA are circularized using CircLigase to form TTB (Step 6). TTB primers can be prepared using the simple PCR, nicking, and gel purification steps. The sequence NNNVTNNNVTNNNVTNNNV in FIG. 1 is represented by SEQ ID NO: 33.

FIGS. 2A and 2B show Tandem Twin Barcode verification by Sanger sequencing. (A) Sequencing results. The presence of the TTB consisting of two identical barcodes tandemly located in the same direction in each oligomer molecule was confirmed by cloning and sequencing 40 TTB DNA using high-fidelity Sanger sequencing. 21 twin barcode sequences were shown. (B) An example chromatogram from Sanger sequencing. Sequence identified a TTB marked with *. SEQ ID NO: 33 is depicted in 2A.

FIGS. 3A and 3B show TTB labeling and PCR amplification of near full-length HIV-1 DNA. (A) A schematic view of TTB labeling and extension. Test TTB primers (light blue arrows with twin triangles) were bound on the Nef gene downstream of the right LTR of the HIV-1 genome (grey line) and extended toward the left LTR by a single cycle PCR. Unbound TTB were removed by Exo-1 digestion followed by heat-inactivation and DNA purification using a PCR purification kit. TTB labeled HIV-1 DNA were PCR-amplified using three different sets of primers (green arrows) amplifying 8.4K, 5.6K, and 2.5K bp HIV-1 DNA fragments. (B) Agarose gel electrophoresis. 8.4K, 5.6K, and 2.5K PCR products from PCR amplification of TTB-labeled HIV-1 DNA are shown on the right side of the gel (TTB PCR). As a control, PCR product amplifying both the TTB-labeled and non-labeled HIV-1 DNA using a common primer binding the Nef site and the 8.4K PCR primer are shown on the left side of the gel. No template control (NTC) and 1Kb DNA ladders are shown as well.

FIGS. 4A and 4B shows long-range sequencing using a Minion device (R7 version). Both lambda bacteriophage DNA digested with BamH1 (average 10K bp) and TTB-labeled HIV-1 DNA PCR products (8.4K bp) were used to test the R7 version Minion sequencer. (A-B) Histogram for read length analysis for Lambda DNA and TTB-labeled HIV-1 DNA sequencing. Read length and total number of read are shown on horizontal- and vertical-axis, respectively. Average read length for the lambda DNA was about 10K bp (in 2D sequencing), with the longest showing a 54K bp (A). Average read length for TTB-labeled HIV-1 DNA was about 6K with the longest showing a 18K bp in 2D sequencing (=8.4K bp TTB-labeled HIV DNA) (B). About 10% channels were available for the R7 version Minion Chip used in these analyses.

FIG. 5A-E shows accurate sequencing and quantification of HIV-1 variants using tandem, twin barcode (TTB) technology and MinION sequencing. (A) A first-generation test TTB library in which each primer has two identical barcode sequences placed next to each other in the same direction was prepared (FIG. 1). TTB primers specific to the nef gene of HIV-1 (NL4.3) were annealed to proviral DNA and extended by a two-step procedure using various thermostable DNA polymerases, including: Phusion-HF (for initiation), Pfu, and a Takara LA PCR kit (for full-extension). (B) TTB-labeled DNA were PCR-amplified for 3 Kbp, 6 Kbp, and 8.5 Kbp products using a Takara LA PCR kit. (C) After sequencing an 8.5 Kbp PCR product using a MinION device, over 155,000 reads were obtained. The histogram shows the read count over the read length. (D) Four identical barcodes in each read were cross-compared to correct barcode read errors. (E) Sequences with an identical barcode were cross-compared to generate a consensus sequence.

FIG. 6 shows sensitive, absolute quantification of target populations. Traditional Sanger sequencing can only identify viral variants with >20% frequency. Single barcoding approaches using NGS sequencing, while highly sensitive, generate significant false-positive data due to sequencing errors. Read number cut-off can remove false-positive data, but it also removes low-frequency true variants. By contrast, TTB molecules of the present invention enables accurate genotyping, and absolute quantitation by enumerating the unique barcodes associated with a given subpopulation.

FIG. 7A-C shows TTB-mediated error-correction generated error-free consensus sequences. (A). After sequencing TTB-labeled, full-length HIV-1 DNA (8.5 Kb PCR product) using a MinION device, over 155,000 reads were obtained. The histogram shows the read count over the read length. (B) A total of 28 sequences for TTB #21 and the reference sequences (bottom row) were subjected to Multiple Sequence Alignment (MSA) to generate a consensus sequence (top row) using MUSCLE and the HIV consensus finder (LANL.gov). Nucleotide positions from 8422 to 8395 are shown. Read accuracy was improved from 80-90% for raw data to 99.32% for the TTB #21 consensus sequence. The sequences shown in the table are, in sequential order, SEQ ID NOS: 34-64. (C) Homopolymer mismatch correction significantly improved the matching ratios. (i & ii) Two mismatch types—ambiguous reads/substitutions (i) and homopolymer mismatches (ii)—in each TTB-DNA consensus sequence were plotted over the sequencing depth for each TTB-DNA. The number of homopolymer mismatches remained ≥47 events regardless of the sequence depth. (iii & iv) Percent matching for each TTB-DNA improved 10-fold after correcting homopolymer mismatches. A 99.99% match was achieved with a sequencing depth of 96.>99.70% accuracy was achieved with a sequence depth of 17-20.

FIG. 8A-B shows a new digital PCR assay to assess the efficiency of TTB labeling. (A) An extension of a TTB primer (orange bent arrow) synthesizes a new DNA strand. The primer binding and initiation of extension (i) and full-length extension (ii) efficiencies are quantified using digital PCR. To test (i), new-strand-specific (FAM) and template-specific (VIC-1) taqman probes are used. The efficiency of (ii) is measured using the FAM probe and another probe (VIC-2) that binds to near the opposite end of the DNA strands, after denaturing the template. (B) PfuUltra and PhusionHF polymerases were compared for (i) using proviral DNA from 30% infected (GFP+) 293T cells. The efficiency was ˜23% for PfuUltra and ˜28% for Phusion-HF: =#green/[(#green+#red)/2]. Greens are FAM and VIC double positive. Infected DNA without TTB primer extension was used for the Negative control (NC). A low level of TTB-TTB interaction (blues: FAM single) was observed.

FIG. 9A-B shows measuring the efficiency of PCR recombination rates during long-range PCR. (A). PCR recombination can occur when primer extensions fail before reaching the other end and anneal to another template to complete their full extension (i to iii). The recombination events can be measured by comparing the two unique barcodes labeled at both 5′ and 3′ ends. (B) PCR efficiencies for different lengths of DNA within the HIV-1 genome is tested using a modified two-end barcoding (duplex) method.

FIG. 10 shows that the efficiency of generating twins can be 83-92%.

FIG. 11A-B shows methods of optimizing long-range droplet PCR. (A) A PCR reaction mixture was partitioned into approximately 20,000 of 1 nL droplets in each PCR tube using a QX200 droplet generator (Bio-Rad). Monolayer droplet image, taken by a LUNA cell counter (Logos Biosystems), shows evenly sized droplets. (B) The efficiencies of 1.7 Kb, 3 Kb, 6 Kb, and 9 Kb HIV-1 DNA PCR were tested with different reaction conditions. Superior amplification results were observed for 3Kb, 6Kb, and 9Kb PCR when using a modified Bio-Rad ddPCR premix (“A” material: see the lane with an asterisk).

FIG. 12 shows TTB adaptors used to improve 1D2 data analysis. TTB-containing adaptor DNA is generated by our TTB library procedure. The TTB adaptor can be used in Nanopore 1D² library preparation procedures to uniquely label individual PCR amplicons.

### DETAILED DESCRIPTION

Definitions

The term “subject” refers to any individual who is the target of administration or treatment. The subject can be any animal, invertebrate or vertebrate. For example, the subject can be a mammal. Thus, the subject can be a human or veterinary patient. The term “patient” refers to a subject under the treatment of a clinician, e.g., physician. The subject can be either male or female.

The term “biological sample” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The terms “peptide,” “protein,” and “polypeptide” are used interchangeably to refer to a natural or synthetic molecule comprising two or more amino acids linked by the carboxyl group of one amino acid to the alpha amino group of another.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

As used herein, the term “barcode” refers to a unique oligonucleotide sequence that allows a corresponding nucleic acid base and/or nucleic acid sequence to be identified. In certain aspects, the nucleic acid base and/or nucleic acid sequence is located at a specific position on a larger polynucleotide sequence (e.g., a polynucleotide covalently attached to a bead). In certain embodiments, barcodes can each have a length within a range of from 4 to 150 nucleotides. The barcode technology (or barcoding) has been a particularly powerful technique for studying the genetic and functional variations of the target pool and for high-accuracy target DNA sequencing. Barcode technologies are known in the art and are described in Verovskaya, E. et al. Blood (2013) 122; Brady, T. et al. (2011) Nucleic Acids Research 39, e72; Naik, S. H., et al., (2014) Experimental Hematology 42, 598:608; Jabara, C. B. et. al., (2011) Proc. Natl. Acad. Sci. 108, 20166; Lee, D. F., et. al. (2016) Nucleic Acids Research, 44, e118; Schmitt, M. W. et al. (2015), Nat. Meth. 12, 432; Kinde, I., et al., (2011) Proc. Natl. Acad., Sci. 108; 9530; Hiatt, J. B., et al., (2013) Genome Research 23, 843; Schmitt. M. W. et al., (2012) Proc. Natl. Acad. Sci. 109, 14508; Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240.

By “tandem, twin barcode (TTB) molecules” is meant two barcodes that are identical to each other (“twins”), and near each other (“tandem”) on a nucleic acid, so that they are contiguous with each other. The TTB molecule comprises a first and second nucleic acid sequence. These two sequences are identical to each other, meaning that they consist of the exact same nucleotides in the same order, so that they are replicas of one another. Each of the first and second nucleic acid sequence of the TTB can be comprises of repeated barcode blocks, described below. The TTB molecules can be flanked by other nucleic acids, such as target nucleic acid. They can be in a molecule that is circularized, or they can be linear. There can be a space between the first and second nucleic acid sequence of the TTB, so that they are joined by a spacer or a linker. This spacer or linker can be comprised of nucleic acid sequences.

By “barcode block” is meant a short nucleic acid sequence that can be used to prevent the formation of a homopolymer longer than 5 nucleotides in a barcode: in a barcode block, three consecutive degenerate nucleotides (Ns) are flanked by nucleotide sequences that prevent homopolymer formation. Each block can be repeated in the first and second nucleic acid, so that a first nucleic acid sequence of the TTB comprises 2, 3, 4, 5, 6, 7, 8, 9, 10, or more repeated blocks. Of course, the second nucleic acid sequence of the TTB, which is identical to the first nucleic acid sequence, will comprise the same sequence of repeated barcode blocks. Examples of such blocks, and their repeat units, can be seen in Table 2.

By “TTB library” is meant multiple TTB molecules present in the same solution. Each TTB molecule can be unique, in that it is different from any other TTB sequence in the library.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Hybridization” refers to the process in which two single-stranded oligonucleotides bind non-covalently to form a stable double-stranded oligonucleotide. The term “hybridization” may also refer to triple-stranded hybridization. The resulting (usually) double-stranded oligonucleotide is a “hybrid” or “duplex.”

“Amplifying” includes the production of copies of a nucleic acid molecule of the array or a nucleic acid molecule bound to a bead via repeated rounds of primed enzymatic synthesis. “In situ” amplification indicated that the amplification takes place with the template nucleic acid molecule positioned on a support or a bead, rather than in solution. In situ amplification methods are described in U.S. Pat. No. 6,432,360.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 100 nucleotides, and more usually. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

“Sequencing” refers to determining the order of nucleotides (base sequences) in a nucleic acid sample, e.g. DNA or RNA. Many techniques are available such as Sanger sequencing and High Throughput Sequencing technologies (HTS). Sanger sequencing may involve sequencing via detection through (capillary) electrophoresis, in which up to 384 capillaries may be sequence analysed in one run. High throughput sequencing involves the parallel sequencing of thousands or millions or more sequences at once. HTS can be defined as Next Generation sequencing, i.e. techniques based on solid phase pyrosequencing or as Next-Next Generation sequencing based on single nucleotide real time sequencing (SMRT). HTS technologies are available such as offered by Roche, Illumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Each of these sequencing technologies have their own way of preparing samples prior to the actual sequencing step. These steps may be included in the high throughput sequencing method. In certain cases, steps that are particular for the sequencing step may be integrated in the sample preparation protocol prior to the actual sequencing step for reasons of efficiency or economy. For instance, adapters that are ligated to fragments may contain sections that can be used in subsequent sequencing steps (so-called sequencing adapters). Or primers that are used to amplify a subset of fragments prior to sequencing may contain parts within their sequence that introduce sections that can later be used in the sequencing step, for instance by introducing through an amplification step a sequencing adapter or a capturing moiety in an amplicon that can be used in a subsequent sequencing step. Depending also on the sequencing technology used, amplification steps may be omitted.

“Multiplex sequencing” refers to a sequencing technique that allows for processing a large number of samples on a high-throughput instrument. For multiplex sequencing, individual “barcode” sequences are added to each sample so that nucleotide sequences from different samples can be distinguished by the unique barcode sequences embedded in each sample. With this technique, multiple DNA or RNA samples can be pooled, processed, sequenced, and analyzed simultaneously.

“2D sequencing” or “1D2 sequencing” refers to a sequencing technology that enables reading both the sense and anti-sense strands (also known as template and complementary strands) in the single-molecule sequencing technologies, including the Nanopore Sequencing technology (Oxford Nanopore Technologies).

As used herein, a “dataset” is a set of data associated with a barcode or set of barcodes. Such data can include nucleotide sequences, as well as data for physical characteristics of a barcode or set of barcodes, such as primary sequence, homology to other sequences, melting temperature, GC content, propensity to form a hairpin, among other distinguishing characteristics or parameters. A dataset may be determined experimentally, calculated, or derived from information in other databases or publications.

As used herein, the term “alignment” refers to the identification of regions of similarity in a pair of sequences. For example, barcode sequences can be aligned, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), among others.

As used herein, a “sequencing read” refers to a sequence of nucleotides generated by sequencing a target nucleic acid.

As used herein, a misidentification error rate refers to the rate at which a barcode sequence fails to be uniquely and correctly identified.

As used herein, an “estimated” error rate refers to the probability of error determined by calculation based on an error model or by sampling from empirical or simulation results based on an error model.

General

Barcodes are sequences incorporated into DNA molecules and can be used to identify the target sample from which the DNA was taken or the target DNA in the sequence data. Incorporating a distinct barcode for each of many samples allows for the pooling and parallel processing of the target samples (or DNA molecules) for various purposes, including studying the genetic and functional variations of a heterogeneous pool; sequencing individual target DNA with reduced read-error rates; and quantifying mixed target polynucleotide molecules. Disclosed herein are tandem, twin barcode sequences and methods for generating sets of tandem, twin barcode sequences useful for improving the accuracy of identifying the target samples (or DNA molecules) by eliminating barcode-read errors and thereby improve such barcoding-mediated DNA sequencing studies. Different sets of barcodes can be tailored for specific sequencing platforms and the number of samples to be processed in parallel. The TTB-mediated sequencing strategy is designed to radically improve the accuracy of reading barcode sequences, thereby improving various barcode-based technologies, including sequencing individual target DNA molecules in mixed population analyses.

Barcoding can be used in a variety of applications in molecular biology. Examples include, but are not limited to, those described in U.S. Pat. No. 7,902,122 and U.S. Pat. Publn. 2009/0098555. Barcode incorporation by primer extension, for example via PCR, may be performed using methods described in U.S. Pat. No. 5,935,793 or US 2010/0227329. In some embodiments, a barcode may be incorporated into a nucleic acid via using ligation, which can then be followed by amplification; for example, methods described in U.S. Pat. Nos. 5,858,656, 6,261,782, U.S. Pat. Publn. 2011/0319290, or U.S. Pat. Publn. 2012/0028814 may be used with the present invention. In some embodiments, multiple barcodes may be used, e.g., as described in U.S. Pat. Publn. 2007/0020640, U.S. Pat. Publn. 2009/0068645, U.S. Pat. Publn. 2010/0273219, U.S. Pat. Publn. 201 1/0015096, or U.S. Pat. Publn. 2011/0257031.

Disclosed herein is a library of tandem twin barcode (TTB) oligonucleotide molecules, wherein said library comprises at least 5 unique TTB oligonucleotide molecules, wherein said TTB molecules comprise a first and second barcode sequence, wherein said first and second barcode sequence are identical to each other and positioned in a same 5′ to 3′ orientation, and wherein said TTB oligonucleotide molecules are flanked on either side by two target regions that are common to all TTB oligonucleotides in the library.

The library of TTB oligonucleotide molecules disclosed herein can comprise 5, 10, 15, 20, 25, 30, 35, 40, 45, 50, 55, 60, 65, 70, 75, 80, 85, 90, 95, 100, 150, 200, 250, 300, 350, 400, 450, 500, 1000, 10,000, 100,000, 10⁵, 10⁶, 10⁷, 10⁸, 10⁹, 10¹⁰, 10¹¹, or more unique tandem twin barcode oligonucleotide molecules. By “unique” is meant that each is different from the others present in the library.

Each individual TTB molecule comprises two sets of nucleic acids, a first and a second nucleic acid. These two nucleic acids are identical to each other, meaning that they have the same sequence. Each of the first and second nucleic acid sequences can be formed from barcode blocks. Each of the first and second nucleic acid of the TTB consists of a unique barcode designed to prevent the formation of long homopolymer nucleotides (identical consecutive nucleotides: e.g. AAAA, TTTT, GGGG, CCCC).

A barcode block can be used to prevent the formation of a homopolymer longer than 5 nucleotides in a barcode: in a barcode block, three consecutive degenerate nucleotides (Ns) are flanked by nucleotide sequences that prevent homopolymer formation. For example:

6 bp Barcode Block:

TNNNVT or TVNNNT (where V = A, G, C);
ANNNBA or ABNNNA (B = G, C, T);
GNNNHG or GHNNNG (H = A, T, C);
CNNNDC or CDNNNC (D = A, T, G);

7 bp Barcode Block:

WSNNNWS or WSNNNSW (W = A, T);
MKNNNMK or KMNNNKM (M = A, C; K = G, T);
RYNNNRY or YRNNNYR (R = A, G; Y = C, T)

Followings prevent from >4 homopolymer nucleotides.

5 bp Barcode Block:

TNNVT or TVNNT (where V = A, G, C);
ANNBA or ABNNA (B = G, C, T);
GNNHG or GHNNG (H = A, T, C);
CNNDC or CDNNC (D = A, T, G);

7 bp Barcode Block:

WSNNWS or WSNNSW (W = A, T);
MKNNMK or KMNNKM (M = A, C; K = G, T);
RYNNRY or YRNNYR (R = A, G; Y = C, T)

Repeat of these blocks in each barcode increases the TTB variation in a library.

The barcode block sequence can be repeated as many times as desired in the TTB (so each of the first and second nucleic acids of the TTB, since they are identical, will contain the same number and type of barcode block).

The TTB molecules can be attached to a target region. The target region can be present on either side of the TTB molecule, or can flank the TTB molecule on both sides. There can be a spacer of any length between the TTB molecule and the target regions. The target region can be capable of ligating or annealing to a target nucleic acid in order to associate the TTB molecule with a target nucleic acid. This can be done in order to sequence, multiplex, or amplify, or in any other way manipulate, the target nucleic acid.

Also disclosed herein is a method of labeling target polynucleotide molecules with a unique identifier, the method comprising labeling the barcode library with target polynucleotide molecules. For example, the target polynucleotide can be sequenced after labeling with the barcode library.

Also disclosed is a kit for labelling a target nucleic acid for sequencing, wherein the kit comprises a) a library of at least 5 unique TTB molecules, wherein said TTB molecules comprise a first and second barcode sequence, wherein said first and second barcode sequence are identical to each other; and b) reagents for sequencing. The kit can comprise various molecular biology reagents, including DNA polymerases, RNA polymerases, Reverse-transcriptases, DNA ligases, RNA ligases, transposases, viral integrase, CRISPR/Cas9, zinc finger nucleases, transcription activator-like effector nucleases, exonucleases, endonucleases, Polynucleotide Kinases, or nucleotides.

Disclosed herein is a method of making a tandem twin barcode (TTB) molecules comprising: (a) providing single, barcoded oligomers; (b) ligating single, barcoded oligomers to form a circularized, single barcoded oligomer; (c) synthesizing a complementary strand of the circularized, single barcoded oligomer to form two barcoded oligomers, where one is a sense strand and one is an antisense strand; (d) nicking 5′ upstream of both sense and antisense oligomers so that each barcode region of sense and antisense oligomers are now single-stranded; (e) synthesizing single-stranded regions of both sense and antisense oligomers to fill in barcoded regions, thereby creating a double-barcoded region on both the sense oligomer and the antisense oligomer; (f) nicking the antisense oligomer in order to differentiate sense and antisense strands oligomer, so that the antisense oligomer is shorter than the sense oligomer; (g) isolating the sense oligomer by denaturation of nicked molecules followed by separation of sense and antisense strands; and (h) circularizing single-stranded, double-barcoded oligomers, thereby forming tandem, twin barcode molecules.

Also disclosed herein is a method of sequencing individual nucleic acid molecules in a sample comprising a plurality of nucleic acid molecules, the method comprising: (a) labeling individual target nucleic acid molecules by annealing, synthesizing, inserting, or ligating tandem, twin barcode oligonucleotide molecules to the 3′ end of the target nucleic acid molecules, thereby creating a barcoded, sense-stranded nucleic acid molecule; (b) using primers specific to the bound tandem, twin barcoded nucleic acid molecules to produce amplicons with a tandem, twin barcode molecule embedded therein; (c) sequencing each amplicon with a tandem, twin barcode molecule embedded therein to produce individual sequence reads; (d) cross-comparing the tandem, twin barcode molecules within a same sequence read to correct any read errors within the barcodes; (e) grouping of nucleic acid sequence reads with identical barcodes; (f) resolving errors in sequencing by forming a consensus of correct nucleic acid sequences; and g) determining correct nucleic acid sequence for each individual nucleic acid molecule.

Disclosed herein is a method of counting nucleic acid molecules in a sample, wherein the sample comprises multiple, different nucleic acids, the method comprising: a) attaching a TTB oligonucleotide molecule to each of the plurality of nucleic acid molecules in the sample to produce a plurality of differently barcoded nucleic acid molecules, wherein said TTB molecules comprise a first and second barcode sequence, wherein said first and second barcode sequence are identical to each other and positioned in a same 5′ to 3′ orientation, and wherein said TTB oligonucleotide molecules are flanked on either side by two target regions; and b) amplifying the plurality of differently barcoded nucleic acid molecules in the sample to produce amplicons of the plurality of differently TTB barcoded nucleic acid molecules.

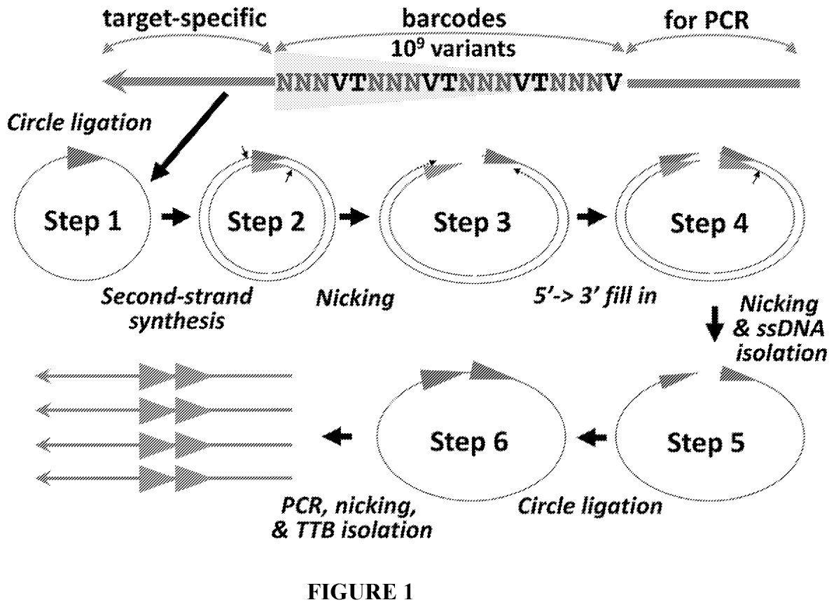

A scheme for preparing TTB molecules can be seen in FIG. 1. The sets of barcodes described herein are typically suitable for a certain sequencing technology. No DNA sequencing technology has perfect fidelity, so that when a DNA sequence is “read”, there will be sequencing errors, and the sequencing errors are characteristic of the sequencing technology used. These sequencing errors can lead to the misidentification of a barcode sequence, i.e., where the actual barcode sequence being read is incorrectly identified as a different barcode. Because the characteristic sequencing errors vary from technology to technology, the estimated performance of a particular set of barcodes will vary by technology. If a given technology has a generally lower sequencing error rate than the technology for which a set of barcodes is “suitable”, the performance of the barcode set will be superior. The estimated worst-case performance of the set of barcodes can be determined from the characteristic errors of the sequencing technology and analysis. If a given technology has a generally higher sequencing error rate than the technology for which a set of barcodes is “suitable”, the performance of the barcode set will be inferior. The estimated worst-case performance of the set of barcodes can again be determined from the characteristic errors of the sequencing technology and analysis.

The TTB molecules disclosed herein can comprise or consist of deoxyribonucleotides. One or more of the deoxyribonucleotides may be a modified deoxyribonucleotide (e.g. a deoxyribonucleotide modified with a biotin moiety or a deoxyuracil nucleotide). The TTB molecules may comprise one or more degenerate nucleotides or sequences. The TTB molecules may not comprise any degenerate nucleotides or sequences. The barcode regions may uniquely identify each of the barcode molecules. Each oligonucleotide molecule that incorporated barcodes may also comprise a sequence that identifies the target sequence. For example, this sequence may be a constant region shared by all barcode regions of a target sequence. Each barcode region may comprise at least 5, at least 10, at least 15, at least 20, at least 25, at least 50 or at least 100 nucleotides. Preferably, each barcode region comprises at least 5 nucleotides.

Each barcode region can comprise deoxyribonucleotides, optionally all of the nucleotides in a barcode region are deoxyribonucleotides. One or more of the deoxyribonucleotides may be a modified deoxyribonucleotide (e.g. a deoxyribonucleotide modified with a biotin moiety or a deoxyuracil nucleotide). The barcode regions may comprise one or more degenerate nucleotides or sequences. The barcode regions may not comprise any degenerate nucleotides or sequences.

The TTB molecules may comprise a linker region between the twin nucleic acids. The linker region may comprise one or more contiguous nucleotides that are not annealed to the target nucleic acid. Alternatively, the linker may be complementary to the target nucleic acid. The linker may comprise 1, 2, 3, 4, 5, 6, 7, 8, 9, 10, 15, 20, 25, 30, 40, 50, 60, 70, 80, 90, 100, 200, 300, 400, or 500 non-complementary nucleotides, or any range or specific number in-between or above this number.

The TTB molecules may be linked by attachment to a solid support (e.g. a bead). For example, barcode molecules of known sequence may be linked to beads. A solution of soluble beads (e.g. superparamagnetic beads or styrofoam beads) may be functionalized to enable attachment of two or more TTB molecules. This functionalization may be enabled through chemical moieties (e.g. carboxylated groups), and/or protein-based adapters (e.g. streptavidin) on the beads. The functionalized beads may be brought into contact with a solution of barcode molecules under conditions which promote the attachment of two or more barcode molecules to each bead in the solution. Optionally, the barcode molecules are attached through a covalent linkage, or through a (stable) non-covalent linkage such as a streptavidin-biotin bond, or a (stable) oligonucleotide hybridization bond.

By “improving accuracy” is meant that there is a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% improvement in accuracy in reading a nucleotide sequence of a single nucleotide molecule compared with a method of sequencing which does not make use of the TTB method disclosed herein. It is noted that the accuracy of the TTB method can be up to 99.9% accurate.

Every barcode in a set can be unique, that is, any two barcodes chosen out of a given set will differ in at least one nucleotide position. Every barcode in a set can be unique, that is, any two barcodes randomly chosen out of a given set will differ in at least one nucleotide position. The random barcode sequences are designed to maximize sequence variations while minimizing read errors. For example, certain sets of barcodes incorporated into DNA or cDNA for sequencing with the Nanopore sequencing technology will have SEQ ID NO: 33 (NNNVTNNNVTNNNVTNNNV) with V(A or C or G) and T repeating every 5 bases. The presence of “VT” at every 5th base in random barcode sequences prevents the formation of consecutive, identical nucleotides (homopolymers) with a length of 5 bp or longer, which frequently induces read errors in Nanopore sequencing. Moreover, the presence of a known nucleotide at a known position can be used as a checkpoint to further improve accurate barcode reading.

In a TTB molecule, the unique barcode is repeated in the “twin” aspect, and the two repeats are adjacent to each other on the molecule, as can be seen in the schematic of FIG. 1. Each set includes at least one unique barcode, repeated as a “twin,” for each sample desired to be processed in parallel, and preferably no more. If it is not known how many samples (or polynucleotide molecules) will be processed in parallel, but if an upper limit on the number of target samples (or polynucleotide molecules) that will be processed is known, then the set of barcodes must exceed the upper limit of the number of samples by at least several magnitudes to ensure that each target sample will be labeled with a unique barcode. For example, if the maximum number of samples to be processed in parallel is 10³ to 10⁴ molecules, a TTB library with SEQ ID NO: 33 (NNNVTNNNVTNNNVTNNNV) that has approximately 10⁹ variations will enable effective analysis of these molecules with unique barcoding rates of 99.69% to 96.51%, respectively (Table 2).

Each barcode sequence in the set satisfies certain biochemical properties that depend on how the set will be used. For example, certain sets of barcode primers will have certain sets of random barcode sequences flanked by a target-specific sequence at the 3′-end and a unique sequence for PCR amplification at the 5′-end, or a target-specific sequence at the 5′-end and a unique sequence for PCR amplification at the 3′-end (FIG. 1). Certain sets satisfy other biochemical properties imposed by the requirements associated with the processing of the DNA molecules into which the barcodes are incorporated. In other words, the barcoded oligomer that is used as starting material can comprise any nucleic acid that is useful for its desired purpose. An example is SEQ ID NO: 33 (NNNVTNNNVTNNNVTNNNV).

TABLE 1
Comparison of maximum read length, throughput, and error rates of DNA
sequencing platforms

| | | Max Read | | Error |
|---|---|---|---|---|
| | Sequencing | Length | Throughput | rates |
| | Platforms | (bp) | (# of reads) | (bp) |
| Conventional | Capillary | ~800 | ~96 | 1/100 |
| | Sequencing | | | |
| “Next- | 454 pyro- | ~700 | ~700,000 | 1/100 |
| generation” | sequencing | | | |
| | (Roche) | | | |
| | Ion Torent | ~400 | ~60,000,000 | 1/100 |
| | (Thermo Fisher) | | | |
| | Solexa (Ilumina) | ~300 | ~375,000,000 | 1/1000 |
| | Solid (Thermo | ~100 | ~260,000,000 | 1/50 |
| | Fisher) | | | |
| “3rd | SMRT (PacBio) | ~14,000 | ~40,000 | 1/100 |
| generation” | Nanopore | several | 20,000 − 800,000* | 1/20- |
| | (Oxford) | 100,000 | | 30** |

*For 2-day run: 20K by MinION and 800K by PromethION: longer run is possible (Run until sufficient).
**with R9 MinION

TABLE 2
Barcode sequence examples

| Seq Id for | | | | Total |
|---|---|---|---|---|
| Repeated | Repeat | | Unit | Vari- |
| Unit | Unit | Barcodes | Repeat | ants |
| NO 1 | NNNVT | NNNVTNNNVTNNNVTNNNVT | 4 | 1.E+09 |
| NO 2 | NNNBA | NNNBANNNBANNNBANNNBA | 4 | 1.E+09 |
| NO 3 | NNNHG | NNNHGNNNHGNNNHGNNNHG | 4 | 1.E+09 |
| NO 4 | NNNDC | NNNDCNNNDCNNNDCNNNDC | 4 | 1.E+09 |
| NO 5 | TVNNN | TVNNNTVNNNTVNNNTVNNN | 4 | 1.E+09 |
| NO 6 | ABNNN | ABNNNABNNNABNNNABNNN | 4 | 1.E+09 |
| NO 7 | GHNNN | GHNNNGHNNNGHNNNGHNNN | 4 | 1.E+09 |
| NO 8 | CDNNN | CDNNNCDNNNCDNNNCDNNN | 4 | 1.E+09 |
| NO 9 | NNNVT | NNNVTNNNVTNNNVT | 3 | 7.E+06 |
| NO 10 | NNNBA | NNNBANNNBANNNBA | 3 | 7.E+06 |
| NO 11 | NNNHG | NNNHGNNNHGNNNHG | 3 | 7.E+06 |
| NO 12 | NNNDC | NNNDCNNNDCNNNDC | 3 | 7.E+06 |
| NO 13 | TVNNN | TVNNNTVNNNTVNNN | 3 | 7.E+06 |
| NO 14 | ABNNN | ABNNNABNNNABNNN | 3 | 7.E+06 |
| NO 15 | GHNNN | GHNNNGHNNNGHNNN | 3 | 7.E+06 |
| NO 16 | CDNNN | CDNNNCDNNNCDNNN | 3 | 7.E+06 |
| NO 17 | NNNVT | NNNVTNNNVT | 2 | 4.E+04 |
| NO 18 | NNNBA | NNNBANNNBA | 2 | 4.E+04 |
| NO 19 | NNNHG | NNNHGNNNHG | 2 | 4.E+04 |
| NO 20 | NNNDC | NNNDCNNNDC | 2 | 4.E+04 |
| NO 21 | TVNNN | TVNNNTVNNN | 2 | 4.E+04 |
| NO 22 | ABNNN | ABNNNABNNN | 2 | 4.E+04 |
| NO 23 | GHNNN | GHNNNGHNNN | 2 | 4.E+04 |
| NO 24 | CDNNN | CDNNNCDNN | 2 | 4.E+04 |
| NO 25 | NNNVT | NNNVTNNNVTNNNVTNNNVTN | 5 | 3.E+11 |
| | | NNVT | | |
| NO 26 | NNNBA | NNNBANNNBANNNBANNNBAN | 5 | 3.E+11 |
| | | NNBA | | |
| NO 27 | NNNHG | NNNHGNNNHGNNNHGNNNHG | 5 | 3.E+11 |
| | | NNNHG | | |
| NO 28 | NNNDC | NNNDCNNNDCNNNDCNNNDCN | 5 | 3.E+11 |
| | | NNDC | | |
| NO 29 | TVNNN | TVNNNTVNNNTVNNNTVNNNT | 5 | 3.E+11 |
| | | VNNN | | |
| NO 30 | ABNNN | ABNNNABNNNABNNNABNNNA | 5 | 3.E+11 |
| | | BNNN | | |
| NO 31 | GHNNN | GHNNNGHNNNGHNNNGHNNN | 5 | 3.E+11 |
| | | GHNNN | | |
| NO 32 | CDNNN | CDNNNCDNNNCDNNNCDNNNC | 5 | 3.E+11 |
| | | DNNN | | |

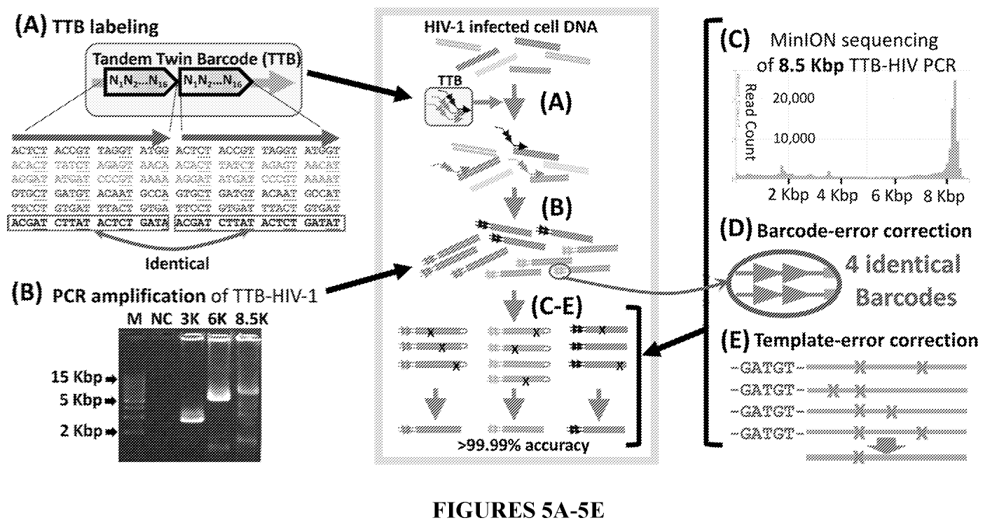

The TTB molecules described above can be used in a method of sequencing, as seen in FIG. 5. The accuracy and read length capacities of the TTB-mediated sequencing technology can provide are vastly superior to the existing technologies, and will have a significant impact on broad areas of genetics and genomics. Therefore, disclosed herein is a method of sequencing individual nucleic acid molecules in a sample comprising a plurality of nucleic acid molecules, the method comprising: (a) labeling individual target nucleic acid molecules with tandem, twin barcode molecules using various methods, including (i) synthesizing an antisense polynucleotide molecule using nucleic acid polymerases and TTB primers that anneal to the 3′ end of the sense-strand of the target nucleic acid molecules, (ii) ligating TTB containing nucleic acid molecules to the target DNA or RNA molecules, or (iii) inserting TTB containing nucleic acid molecules into target DNA or RNA using for example viral integrase, CRISPR/Cas9, or various transposase; (b) removing unbound tandem, twin barcode molecules; (c) using primers specific to the bound tandem, twin barcoded nucleic acid molecules to produce amplicons with a tandem, twin barcode molecule embedded therein; (d) sequencing each amplicon with a tandem, twin barcode molecule embedded therein to produce individual sequence reads; (e) cross-comparing the tandem, twin barcode molecules within a same sequence read to correct any read errors within the barcodes; (f) grouping of nucleic acid sequence reads with identical barcodes; (h) resolving errors in sequencing by forming a consensus of correct nucleic acid sequences; and (i) determining correct nucleic acid sequence for each individual nucleic acid molecule.

“Consensus sequence” is a term of the art that refers to a defined sequence that best represents, statistically, the highest probability of a correct sequence through multiple iterations of sequencing and/or amplification.

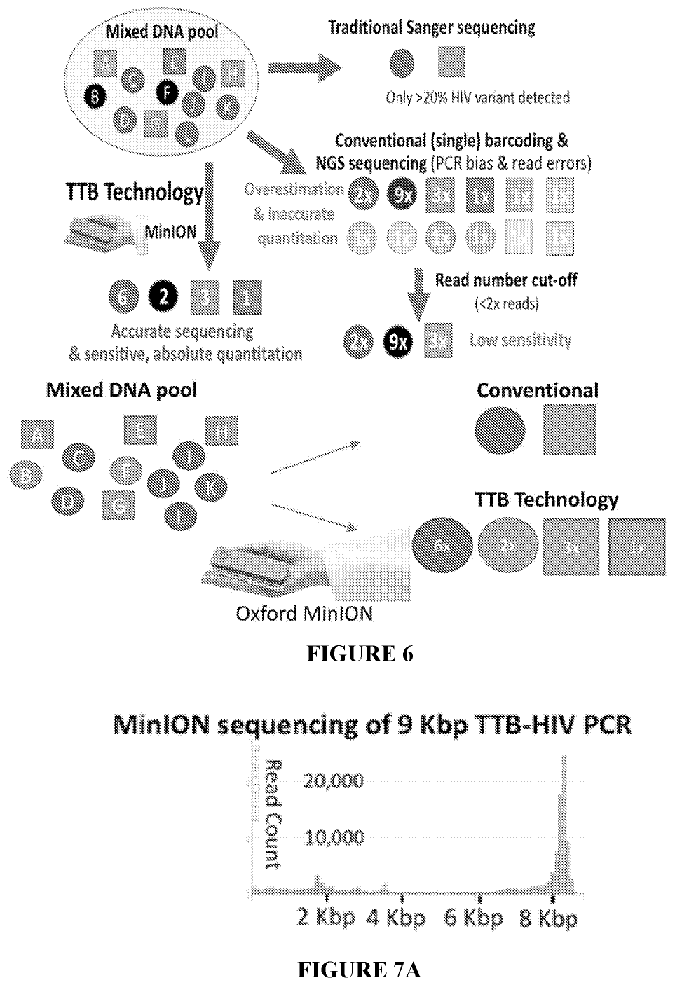

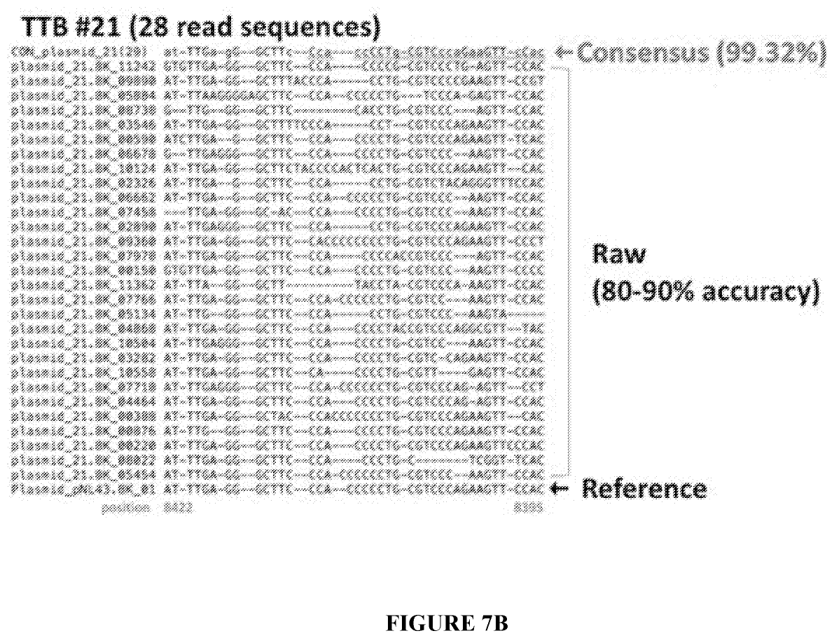

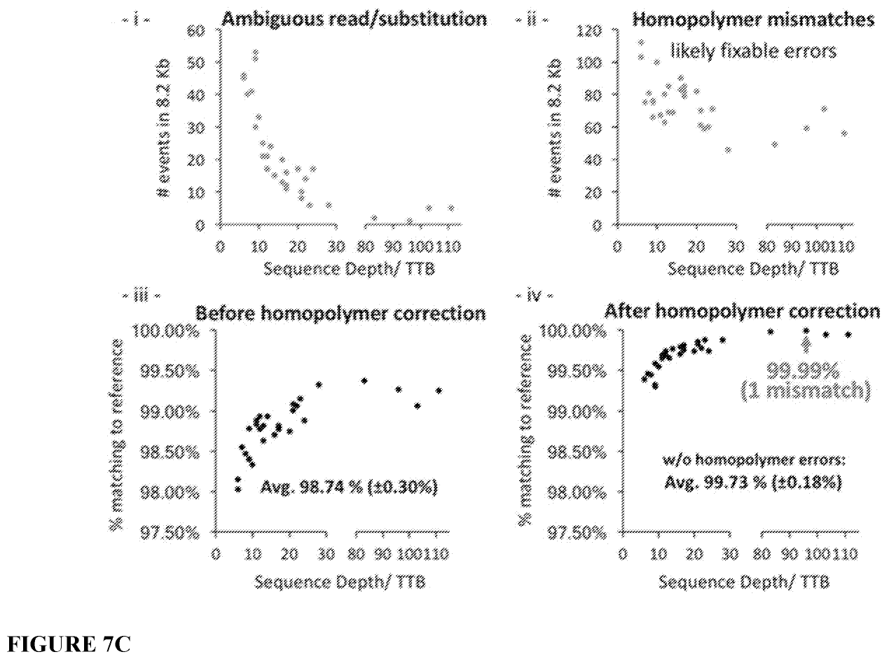

Using the methods of sequencing using TTB as disclosed herein, the relative frequencies of distinct nucleic acid molecules in a mixed pool can be determined. Obtaining accurate quantitative and qualitative information about polynucleotides in a tagged library can result in a more sensitive characterization of the initial genetic material. Typically, individual polynucleotides are amplified and the resulting amplified molecules are sequenced. Depending on the throughput of the sequencing platform used, only a subset of the molecules in the amplified library produce sequence reads. So, for example, the number of amplified molecules sampled for sequencing may be about only 50% of the unique polynucleotides in the PCR amplified pool. Furthermore, amplification may be biased in favor of or against certain sequences. Also, sequencing platforms can introduce errors in sequencing. For example, sequences can have a per-base error rate of 0.5-5%, depending on the sequencing platform. Amplification bias and sequencing errors introduce noise into the final sequencing product. These errors can occur within the barcode sequences or target template DNA sequences. This noise can diminish sensitivity of detection. For example, sequencing 14-20 bp barcodes with NGS (1/100-1/1000 bp error rates) will result in 2-20% error and misidentification rates, while error-prone Nanopore sequencing (currently 1/20 bp error rates) will result in read errors in nearly all barcodes. These errors, including type I (false positive) errors and misidentification of different barcodes (collision), lead to over-estimation, cross-contamination, and erroneous quantification of the barcoded target DNA. Sequence variants whose frequency in the tagged population is less than the sequencing error rate can also be mistaken for noise, thus removing potentially important low-frequency variants from the analysis results (FIG. 6). Furthermore, by providing reads of certain sequences in greater or less amounts than their actual number in a population, amplification bias can distort measurements of copy number variation. This disclosure provides methods of accurately detecting and reading unique polynucleotides in a barcoded (or tagged) pool. In certain embodiments this disclosure provides TTB-tagged nucleic acid that, when amplified and sequenced, can eliminate barcode read errors and provide information that allowed the tracing back, or collapsing, of progeny polynucleotides to the unique tag parent polynucleotide molecule. Collapsing families of amplified progeny polynucleotides reduces amplification bias by providing information about original unique parent molecules. Collapsing also reduces sequencing errors by eliminating from sequencing data mutant sequences of progeny molecules (FIG. 6).

Sequencing of TTB-labeled, PCR amplified polynucleotides generates sequence reads with multiple identical barcodes within the same read (two identical barcodes in each strand; and four identical barcodes in 2D sequencing or 1D² sequencing data, with two in the sense strand and two in the antisense strand), and cross-comparing these multiple identical barcodes in the same read eliminates barcode read errors. This barcode self-error correction effectively reduces the issues associated with barcode-reading errors and thereby deliver critical improvements in barcoding technology.

Detecting and reading unique polynucleotides in the tagged (or barcoded) library can involve two strategies. In one strategy a sufficiently large subset of the amplified progeny polynucleotide pool is a sequenced such that, for a large percentage of unique tagged parent polynucleotides in the set of tagged parent polynucleotides, there is a sequence read produced for at least one amplified progeny polynucleotide in a family produced from a unique tagged parent polynucleotide (this is different than the presently claimed invention). In a second strategy, the amplified progeny polynucleotide set is sampled for sequencing at a level to produce sequence reads from multiple progeny members of a family derived from a unique parent polynucleotide. Generation of sequence reads from multiple progeny members of a family allows collapsing of sequences into consensus parent sequences. These methods can be combined with any of the sequencing noise reduction methods known to those of skill in the art. These include, but are not limited to, qualifying sequence reads for inclusion in the pool of sequences used to generate consensus sequences.

The target nucleic acid molecules can be obtained from an individual cell, non-cellular microorganisms, or synthetic entities. The systems and methods of this disclosure may have a wide variety of uses in the manipulation, preparation, identification and/or quantification of nucleic acid. Examples of nucleic acids include but are not limited to: DNA, RNA, amplicons, cDNA, dsDNA, ssDNA, plasmid DNA, cosmid DNA, high Molecular Weight (MW) DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA (e.g., retroviral RNA).

Nucleic acids that can be used with the methods disclosed herein may be derived from a variety of sources including human, mammal, non-human mammal, ape, monkey, chimpanzee, reptilian, amphibian, or avian, sources. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. The nucleic acids may be fetal in origin (via fluid taken from a pregnant subject), or may be derived from tissue of the subject itself.

In one embodiment, an algorithm can used to resolve errors in sequencing based on multiple sequence reads from the same target nucleic acid molecule labeled with a unique twin barcode. Such algorithms are known to those of skill in the art.

Sequencing can take place by any means known in the art. Examples include, but are not limited to, those described above in the “definitions” section, as well as lab-on-chip technology, microfluidic technologies, biomonitor technology, proton recognition technologies (e.g., Ion Torrent), single cell third generation sequencing (e.g.PacBio™ or Oxford Nanopore MinION™ hand-held sequencer; and other highly parallel and/or deep sequencing methods). When MinION™ is used, it can be any version, including versions 9.4 and 9.5. Also disclosed are conventional Sanger sequencing methods, Sanger capillary sequencing, Solexa™ sequencing (Illumina™, HiSeq™, MiSeg™, NextSeg™, MiniSeg™, and iSeq™), SOLiD™ sequencing, 454 pyrosequencing, SMRT™ (single molecule, real time) sequencing, and Helicos™ single molecule fluorescence sequencing.

In any method of preparing a nucleic acid sample for sequencing, either the nucleic acid molecules within the nucleic acid sample, and/or the TTB molecules, may be present at particular concentrations within the solution volume, for example at concentrations of at least 100 nanomolar, at least 10 nanomolar, at least 1 nanomolar, at least 100 picomolar, at least 10 picomolar, or at least 1 picomolar or less. The concentrations may be 1 picomolar to 100 nanomolar, 10 picomolar to 10 nanomolar, or 100 picomolar to 1 nanomolar. Alternative higher or lower concentrations may also be used.

Each barcoded target nucleic acid molecule may comprise at least 1, at least 5, at least 10, at least 25, at least 50, at least 100, at least 250, at least 500, at least 1000, at least 2000, at least 5000, or at least 10,000 nucleotides synthesized from the target nucleic acid as template. Preferably, each target nucleic acid molecule comprises at least 1 nucleotide synthesized from the target nucleic acid as template. The target nucleic acid may in an intact nucleic acid molecule, co-localized fragments of a nucleic acid molecule, or nucleic acid molecules from a single cell. Preferably, the target nucleic acid is a single intact nucleic acid molecule, two or more co-localized fragments of a single nucleic acid molecule, or one, or two or more nucleic acid molecules from a single cell.

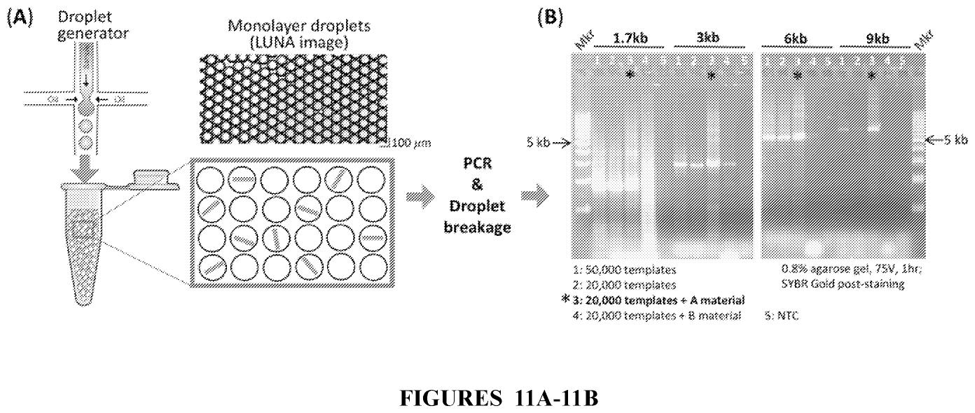

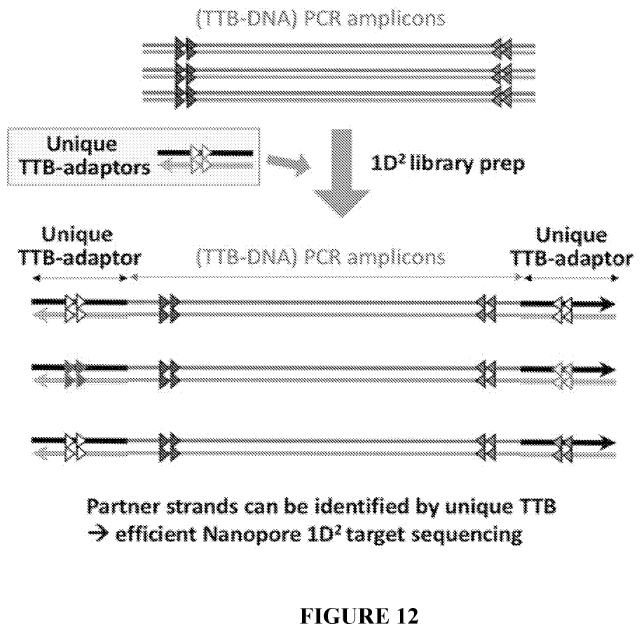

Long range PCR can also be used with the methods disclosed herein. Long-range PCR conditions using 1-3-6-9K PCR can be done, and recombination events can be reduced by modifying PCR conditions; for example, by partitioning a PCR reaction into numerous droplets, some droplets will contain one or more copies of target DNA and some will not contain any targets, and by changing primer concentration, elongation time, amplification cycles, DNA polymerase, or input DNA copies. For example, droplet PCR can be used, as shown in FIG. 11A-B and explained in Example 7.

Also disclosed are computer readable programs and kits compatible with the methods disclosed herein. The invention also provides kits and computer readable programs specifically adapted for performing any of the methods defined herein.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Tandem Twin Barcodes (TTB) and Nanopore Sequencing for Highly Accurate, Quantitative, and Long-Read Sequencing

The TTB-mediated sequencing strategy is designed to radically improve the accuracy of the sequencing individual target DNA molecules in mixed population analyses. HIV-1 genotype variants were focused on as a proof of concept. The sequencing strategy using TTB and the Nanopore Technology can genotype full-length, individual HIV DNA molecules with 99.99% or higher accuracy. Briefly, each of individual target DNA molecules are uniquely labeled with TTB by annealing TTB primers on the Nef (or PBS) region of the HIV-1 (FIG. 5). TTB-labeled DNA is then PCR amplified and processed for Nanopore 2D sequencing. Note that each TTB primer has two identical barcodes next to each other in the same direction. The 2D sequencing technology reads both sense and antisense strands using a loop adaptor, which significantly improves sequencing accuracy up to 85% (R7 version)-95% (R9 version). Thus, each 2D sequence has 4 identical barcodes (two in the sense and two in the antisense) (FIG. 5D). This allows accurate determination of the unique barcode in each 2D sequence: even if sequence errors may have occurred within a barcode, the correct barcode sequence can be readily determined by cross-comparing all 4 barcodes in the same sequence. After determining TTB, the same TTB-labeled sequences are cross-compared to correct any errors in HIV sequence that may have occurred during PCR and/or sequencing. This strategy allows an unprecedented high-accuracy sequencing of the full-length, single HIV-1 DNA molecule (99.99% or higher accuracy: less than one error in the 10 Kb HIV genome).

Several TTB libraries specific to different HIV subtypes and other pathogens, including hepatitis B and C viruses and tuberculosis, can be used for simultaneous analysis of HIV and co-infected pathogens. As each TTB indicates a different target DNA molecule in the sample, absolute quantities of target populations can be measured by counting the number of unique TTBs associated with a given (identical genotype) population. With this approach, even low-frequent variants can be fully sequenced and quantified—an otherwise impossible task with existing means (FIG. 6). Rapid, on-site detection of HIV-1 and co-infected pathogens are crucial in patient care as well as the effective control of diseases.

The accuracy and read length capacities that the TTB-mediated Nanopore sequencing technology can provide are vastly superior to the existing technologies, and have a significant impact on broad areas of genetics and genomics. This technology is particularly useful in genotyping and understanding the dynamics of individual microorganisms and their genetic variants in a mixed pool. Genotypic diversity is a major challenge to the control of a great number of microbial pathogens: not just HIV-1, but any number of microbial pathogens for which antimicrobial drugs are being developed/employed.

This technology is useful for the real-time, on-site monitoring of infectious pathogens thanks to Nanopore technology's pocket-size mobility (MinION) and real-time data output, along with the ongoing development of various molecular biology kits and tools that support sample preparation, sequencing, and data analysis in resource limited settings. In particular, the on-site and real-time detection and genotyping is of crucial importance for the effective control of emerging infectious agents, including Ebola, Zika, influenza, Middle East Respiratory Syndrome (MERS), and antibiotics resistant microbiomes. Furthermore, effective HIV/AIDS prevention and patient care in resource limited settings is the highest priority to end the AIDS epidemic. This technology is a key addition for genotyping and monitoring of HIV variants and co-infected pathogens, as well as other emerging pathogens in resource limited settings.

A platform technology for monitoring HIV-1, and co-infected HCV, HBV, and tuberculosis was developed. TTB libraries are being generated and tested. A TTB primer library specific to HIV-1 DNA has been generated. PCR amplification and the sequencing of full-length TTB-labeled HIV-1 have also been successfully tested using MinION. Sequence analysis pipelines are also being developed.

Major required steps have already been successfully tested using a molecular clone of HIV-1 (pNL4.3). Because TTB, unlike other barcode primers, cannot be chemically synthesized, molecular biology procedures have been developed that generate an HIV-1-specific TTB library with 10⁹ variants. The improved sequencing accuracy of the R9 version of MinION can significantly reduce the minimum required depth of TTB-labeled sequences to achieve greater than 99.99% accuracy genotyping. With R9 sequencing (initial accuracy of 95%), 5 or more copies of HIV-1 sequences are required.

This on-site, real-time pathogen sensing platform technology can significantly improve patient care and disease prevention, particularly in resource limited settings. The radical improvement of sequencing capacity achieved with this technology has a profound impact on sequencing-based scientific research and medicine. Genome sequencing processes are significantly simplified and novel research and diagnostic applications can be developed.

### Example 2

### Preparation of a Tandem Twin Barcode (TTB) Library

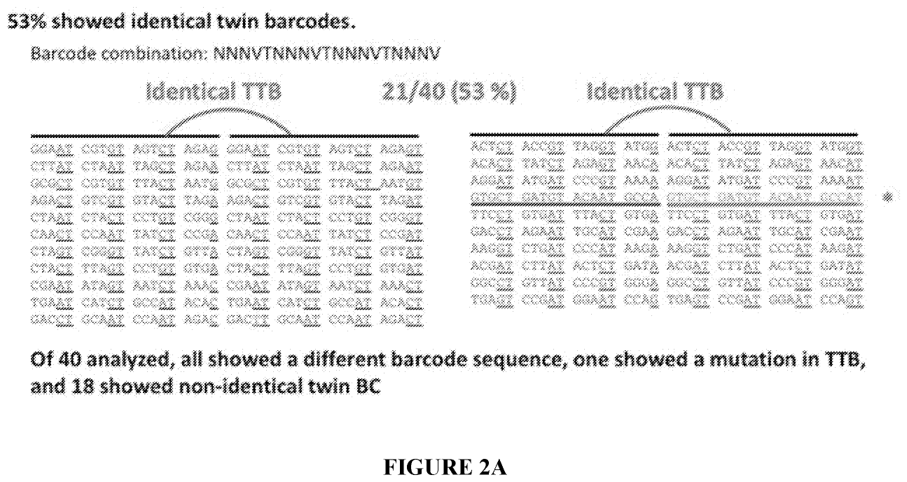

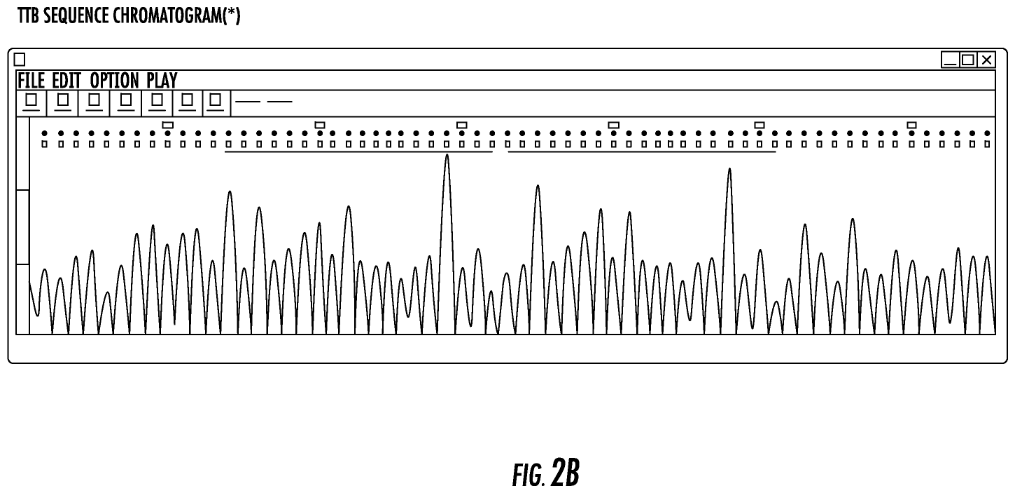

TTB preparation procedures does not utilize polymerase chain reaction (PCR) until Step 6 (FIG. 1), which minimizes potential biases in the barcode combinations associated with PCR. An initial test barcode library has been generated with >10¹¹ DNA molecules without PCR. As a 19-base barcode (NNNVTNNNVTNNNVTNNNV, SEQ ID NO: 33) can accommodate >10⁹ variants, there is approximately 100 DNA copies per barcode in the current library. To test the quality of the TTB library, 40 TTB DNA were cloned and sequenced using high-fidelity Sanger sequencing. Sequencing results showed that all 40 samples had a different barcode sequence; of those, about 53% had identical twin 19 bp-barcodes (FIG. 2). This has since been updated to show that the efficiency of generating twins can be 83-92% efficient, as shown in FIG. 10.

### Example 3

### TTB Labeling and PCR Amplification of Near Full-Length HIV-1 DNA

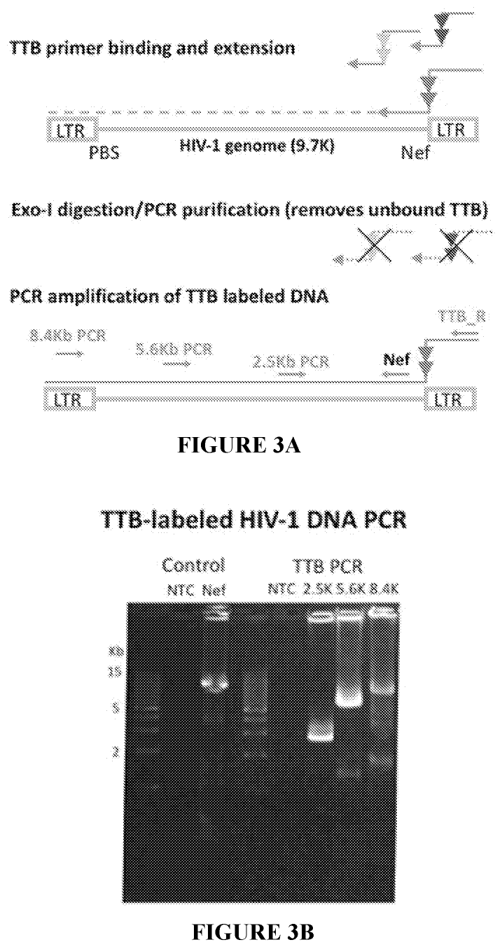

Test TTB primers were used to optimize long-range PCR for HIV-1 DNA (FIG. 3). TTB primers were bound on the Nef gene upstream of the right LTR of the HIV-1 genome (from NL4.3 plasmid DNA and NL4.3 infected T-cells) and extended toward the left LTR using a thermostable DNA polymerase (FIG. 3A). Unbound TTB were removed by Exo-1 digestion followed by heat-inactivation and DNA purification using a PCR purification kit. TTB labeled HIV-1 DNA were PCR-amplified using three different sets of primers amplifying 8.4K, 5.6K, and 2.5K bp HIV-1 DNA fragments. A PCR band of 8.4K bp TTB-labeled, near full-length HIV-1 DNA is shown in FIG. 3B. A PCR result using a primer binding the Nef site—common for both the TTB-labeled and non-labeled HIV-1 DNA—and the 8.4K PCR primer in the left LTR is shown as a control.

### Example 4

### Long-Range Sequencing Using a Minion Device (R7 Version)

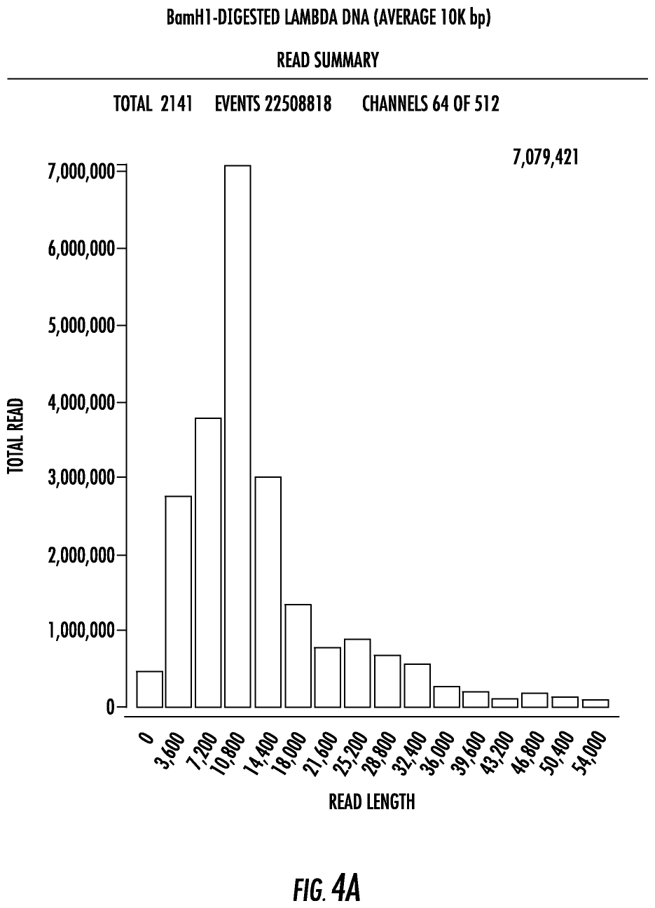

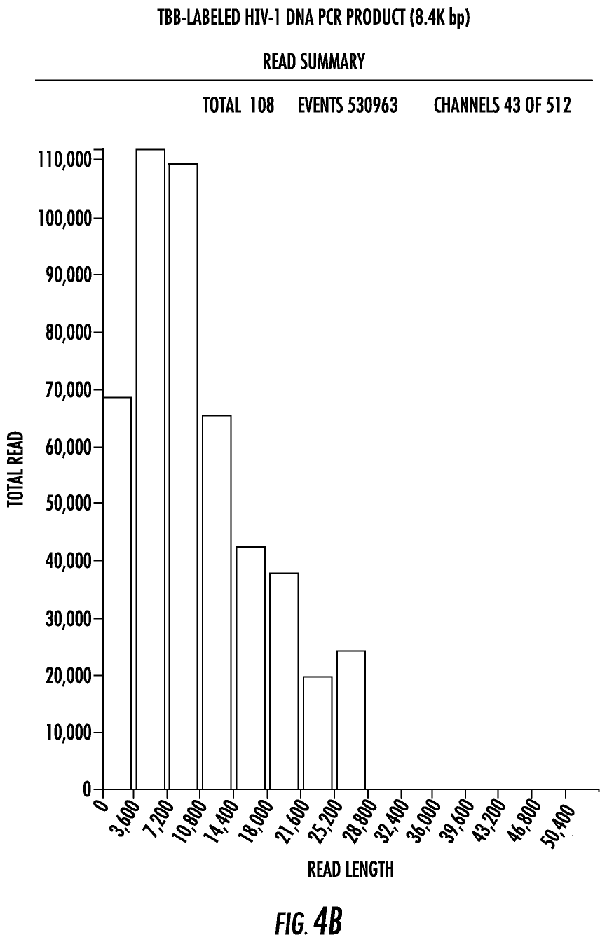

Both lambda bacteriophage DNA digested with BamH1 (average 10K bp) and TTB-labeled HIV-1 DNA PCR products (8.4K bp) were used to test the R7 version Minion sequencer (FIG. 4). Average read length for the lambda DNA was about 10K bp (in 2D sequencing), with the longest showing a 54K bp (FIG. 4A). In TTB-labeled HIV-1 DNA sequencing (FIG. 4B), average read length was about 6K with the longest showing a 18K bp in 2D sequencing (=8.4K bp TTB-labeled HIV DNA). The new R9 version Minion can provide much longer read length and accurate sequence data.

### Example 5

### Preparation Steps for Tandem Twin Barcodes (TTB)

Step 1: intra-molecular circle ligation of barcode primers (10⁹ variants). Step 2: second-strand synthesis using a reverse primer binding the 3′ end of the barcode primers and thermo-stable polymerases.

Step 3: site specific nicking at 5′ upstream of the barcodes of each strand

Step 4: 5′ to 3′ DNA synthesis to fill in the barcode sites.

Step 5: single-strand DNA (ssDNA) isolation using a denaturing acrylamide gel after site-specific nicking of the newly synthesized strand.

Step 6: intra-molecular circle ligation.

Step 7: PCR amplification followed by TTB primer isolation using a denaturing gel.

### Example 6

### Barcoding Technology

What is needed in the art is a novel barcoding technology which can radically improve the accuracy of single-molecule, target DNA sequencing for the Nanopore sequencing platform. Innovations in sequencing technologies over the past decade have been critical driving forces behind the ongoing revolution in medicine and the life sciences. The Nanopore sequencing platform (Oxford Nanopore Technologies) is a newer, so-called third generation sequencing technology with several futuristic features, including an extremely long-read length capacity (the company record: 350 Kbp), real-time data output, and pocket-size mobility (thanks to the MinION sequencer). Given that the growth of sequencing-based research and business opportunities is highly dependent upon the technological strength of available sequencing platforms, the Nanopore sequencing technology is expected to have a highly significant and perhaps revolutionary impact on broad and diverse areas of the sequencing field.

A critical barrier to broader applications of this emerging technology has been its disappointingly high sequencing error rates (approximately 1 out of 20 bases). Barcoding (also known as molecular tagging) has been an excellent tool for reducing erroneously called variants for short-read next-generation sequencing (NGS) platforms. Individual target DNA molecules can be accurately sequenced by barcoding the individual molecules, then amplifying and sequencing the barcoded DNA for the purpose of error correction. The application of current barcoding methods to the error-prone Nanopore sequencing platform is currently impractical because of the high error rates in reading barcodes: for example, when reading 14-20 base pair barcodes, Nanopore sequencing will generate read errors in nearly all barcodes. Disclosed herein is a special twin barcode (Tandem Twin Barcode or TTB) approach that eliminates barcode read errors even in error-prone Nanopore sequencing by enabling cross-comparison of 4 identical barcodes in the same read (2 in the sense strand and 2 in the anti-sense strand) for self-error correction. This accurate barcode reading allows for the effective application of barcoding-mediated error correction approaches, thereby reducing template read errors for Nanopore sequencing. The full-length HIV-1 genome was sequenced using a Nanopore sequencer (MinION, Oxford) and high-accuracy individual HIV-1 DNA sequences with fewer than one error in the 9 Kbp HIV-1 DNA were generated. This is an improvement by several orders of magnitude over others, including Nanopore's average error rates.

Long-range, high-accuracy sequencing is essential in delineating the heterogeneity of cellular or viral populations in diseases such as cancer or viral infection. The HIV-1 genome was genotyped as a proof of principle. HIV-1 is an excellent model system because of the relative small genome of HIV-1 (approximately 9 Kbp), the high degree of intra-patient genetic diversity, and the availability of sufficient NGS and Sanger sequence data for purposes of comparison. Standard experimental procedures for a new-generation TTB library and a self-error correction method using this TTB library were generated, and the utility of the method was tested using laboratory strains of HIV-1.

Radical improvement in the accuracy of long-range, single-molecule sequencing using Nanopore sequencers: Although the low-cost, massively high-throughput sequencing capacities of “next-generation sequencing” (NGS) platforms have already had major impacts on science and medicine, there remains an unmet need for long-read length and low error rates (Table 3). Nanopore sequencing (Oxford Nanopore Technologies) has opened up new avenues for long-range, real-time single-molecule sequencing with a portable and low-cost device, however, its current error rates are disappointingly high, severely limiting its applications. The disclosed TTB-mediated error-correction method improves the accuracy of target molecule sequencing using Nanopore sequencers (Table 3), thereby facilitating the application of the revolutionary features of Nanopore sequencing in broad areas of science and medicine.

TABLE 3
Comparison of maximum read length, throughput, and error rates of DNA
sequencing platforms.

| | | Max Read | | Error |
|---|---|---|---|---|
| | Sequencing | Length | Throughput | rates |
| | Platforms | (bp) | (# of reads) | (bp) |
| Conventional | Capillary | ~800 | ~96 | *1/100 |
| | Sequencing | | | |
| Next- | 454 pyrosequencing | ~700 | ~700,000 | *1/100 |
| generation | (Roche) | | | |
| sequencing | Ion Torent | ~400 | ~60,000,000 | *1/100 |
| (NGS) | (Thermo Fisher) | | | |
| | Solexa (Ilumina) | ~300 | ~375,000,000 | *1/1000 |
| | Solid | ~100 | ~260,000,000 | *1/50 |
| | (Thermo Fisher) | | | |
| 3rd | SMRT (PacBio) | ~14,000 | ~40,000 | *1/100 |
| generation | MinION (Oxford) | several | **200,000 | *1/20 |
| sequencing | | 100,000 | (9 Kbp HIV) | |
| Our | MinION with | >10,000 | **10,000 | **<1/ |
| approach | TTB | (?) | (depth =20) | 10,000 |

*Fox et al. 2015,
**For 2-day run for 9 Kbp HIV-1 DNA using R9.5 MinION; A longer run is possible (Run until sufficient);

Accurate barcode determination using TTB: Barcoding has been a particularly powerful technique for studying the genetic and functional variations of the target pool. Barcoding-mediated error correction methods improve the accuracy of sequencing individual DNA molecules and have been effective for ultrasensitive detection and quantification of genetic variants in cancers or infectious diseases. Although powerful, these approaches have been hindered by frequent type I (false positive) errors in barcode reads and misidentification of different barcodes (collision), leading to over-estimation, cross-contamination, and erroneous quantification of barcoded DNA.14-19 The utility of conventional barcoding approaches greatly depends upon the read accuracy of a given sequencing platform. For example, sequencing 14-20 bp barcodes with NGS (1/100-1/1000 bp error rates) will result in 2-20% error and misidentification rates, while error-prone Nanopore sequencing (currently 1/20 bp error rates) will result in read errors in nearly all barcodes. Our TTB approach, which effectively eliminates these issues via self-error-correction, delivers critical improvements in barcoding, not just for Nanopore sequencing but for any sequencing platform.

Innovations in pathogen genotyping and surveillance. Emerging infectious diseases, like HIV/AIDS, SARS, H1N1 influenza, and Ebola, remain a dire threat to human health and global economic stability. The emergence of infectious pathogens is both unpredictable and inevitable. Furthermore, genotypic diversity and the continual hyper-evolution of microbial pathogens have presented major challenges to the development of countermeasures. Rapid and effective surveillance and diagnosis of emerging pathogens are the keys to the successful control of emerging infectious diseases. The claimed approach makes a groundbreaking impact on this field by providing a means by which to accurately genotype and quantify individual pathogens via on-site, real-time sequencing at the whole genome level, even in a resource-limited setting. A few examples (a-c) follow here. (a) Sensitive detection and quantification of HIV-1 variants. Sensitive detection of drug-resistant mutants is of substantial importance for effective patient care. Current genotyping methods are limited in their ability to detect low-frequency, minority variants and may overestimate them due to sequencing errors. The claimed approach—which analyzes each individual HIV-1 genome separately with TTB—can sensitively detect all sizes of subpopulations and accurately quantify them without any bias by counting the unique barcodes associated with a given subpopulation (FIG. 6). (b) High-throughput, full-length HIV-1 genotyping. Recent full-length HIV-1 genotyping studies have revealed that the majority of integrated HIV-1 DNA in patients' samples have severe deletions/mutations and thus are non-infectious. These dead viruses may complicate the identification of infectious, emerging viral clusters. Conventional genotyping methods—which focus only on the sequencing of the vital enzymes (pol or env gene) of HIV-1 cannot distinguish the dead viruses from full-length, intact viruses. The full-length genotyping assay disclosed herein can significantly improve the ability to identify and monitor emerging viruses and to predict clinical outcomes. The approach can operate at a several-fold higher throughput and rate of accuracy than other comparable methods, including limiting dilution full-length sequencing and single-molecule genome sequencing. Full-length sequencing can also increase the accurate identification of HIV-1 strains in particular recombinant forms, epidemiological signatures and recombinant hot spots and immune recognition at the whole genome level, and nondrug target mutation. (c) Real-time, on-site pathogen surveillance platform: UNAIDS has set ambitious 90/90/90 targets toward ending the HIV/AIDS epidemic: that by 2020, 90% of people living with HIV will know their HIV status; 90% of those diagnosed will be on sustained antiretroviral treatment; and 90% of those on treatment will have achieved viral suppression. Reaching these goals will require precise and efficient testing, patient care, and prevention interventions for key populations, particularly in resource-limited settings. The sequencing platform disclosed herein has the potential to serve as an on-site pathogen surveillance tool, thereby significantly enhancing the ability to (1) sensitively detect and monitor emerging or expanding HIV transmission clusters, (2) predict drug-resistant mutations so as to ensure effective treatment, and (3) improve patient care and retention by enabling on-site genotyping even in resource-limited settings.

Accurate, long-range sequencing using MinION and TTB: Disclosed herein is a TTB-mediated self-error-correction method for the high-accuracy sequencing of individual target DNA molecules. This strategy uses a library of TTB, with two identical barcodes for every target-specific primer (FIG. 5A). The first step was to assign unique TTB to individual target molecules, followed by PCR amplification of TTB-labeled DNA (FIG. 5B). MinION 2D sequencing—which enables sequencing of both the sense and antisense strands—3 generated multiple copies of DNA sequences with identical TTB (FIG. 5C). At this stage, all sequences were expected to have an error rate of approximately 5% (for example, more than 400 errors in each of the 8.5 Kbp HIV-1 sequences). In the next stage, the barcodes for each sequence read were determined by cross-comparing the 4 identical barcodes in each read: 2 in the sense strand and 2 in the antisense strand (FIG. 5D). After determining the barcodes, all sequences with identical barcodes were grouped together (as a family of sequences originating from the same parental DNA molecule) and cross-compared in order to correct sequence errors (FIG. 5E). Multiple TTB-labeled, full-length parental HIV-1 sequences with greater than 99.99% accuracy (less than one error in the whole HIV-1 genome).

Creation of a Tandem Twin Barcode (TTB) library in order to eliminate barcode read errors. Four identical barcodes, available within the same read to enable cross-comparison, eliminates barcode read errors and thereby significantly improve studies using barcoding approaches. A library of TTB primers can be generated that has at least 10⁹ sequence variants and a minimum of 10¹² TTB primer molecules in order to effectively analyze up to 10⁶ different target DNA molecules.

Designing barcode sequences. The 19 bp barcode sequence (FIG. 1) is designed to maximize sequence variations while minimizing read errors. The presence of “VT” (V=A, G, or C) at every 5th base in the random barcode prevents the formation of >5 bp consecutive, identical nucleotides (homopolymers), which frequently induces read errors in Nanopore sequencing. The VT at every 5th base can also be used as a checkpoint to further improve accurate barcode reading.

Generating a tandem twin barcode library. A procedure has been developed that can generate a TTB library from single-barcoded primers (FIG. 1). The initial barcode primers consist of a 19 bp barcode sequence flanked by a target-specific sequence at the 3′-end and a unique sequence for PCR amplification at the 5′-end. The initial barcode primers are chemically synthesized using the “hand mix” option (Integrated DNA Technologies, Inc.) to ensure the even distribution of the four nucleotides at each N position. To generate the twin barcode formation, a molecular biology procedure was developed that mimics the way that retroviruses generate a short duplication of the host DNA during its integration process. The barcode primers are circularized using single-stranded DNA (ssDNA) Ligase (CircLigase, Illumina), double-stranded with a DNA Polymerase, digested with strand-specific endonucleases to separate the sense and anti-sense barcodes, treated with a DNA polymerase in order to synthesize the second strand of the barcodes, and finally re-circularized after the removal of the anti-sense strand. No PCR or cloning steps are used during TTB formation to prevent any potential bias that might result from these steps. After completing the TTB formation, TTB primers can be generated and maintained by simple PCR and ssDNA purification procedures. The quality of the library is assessed by high-throughput illumine sequencing and statistical analysis (capture and recapture and Bayesian method). The efficiency of each step is quantified using digital PCR (Quantstudio 3D, Thermofisher), real-time PCR, and spectrophotometers (NanoDrop, Thermofisher) so as to optimize and standardize the procedures.

Optimize and standardize the TTB labeling and long-range PCR procedures for TTB-mediated long-range sequencing. Previous studies using NGS and barcoding for high-accuracy sequencing have revealed low recovery rates for target DNA, along with template mutation/recombination (occurring during PCR), as major challenges. Disclosed herein are method used for (1) TTB-labeling and (2) long-range PCR steps in order to maximize the sensitivity and efficiency of the platform technology. Novel digital PCR (dPCR) and Nanopore sequencing-based assays that accurately quantify the efficiency of each sub-process are be used

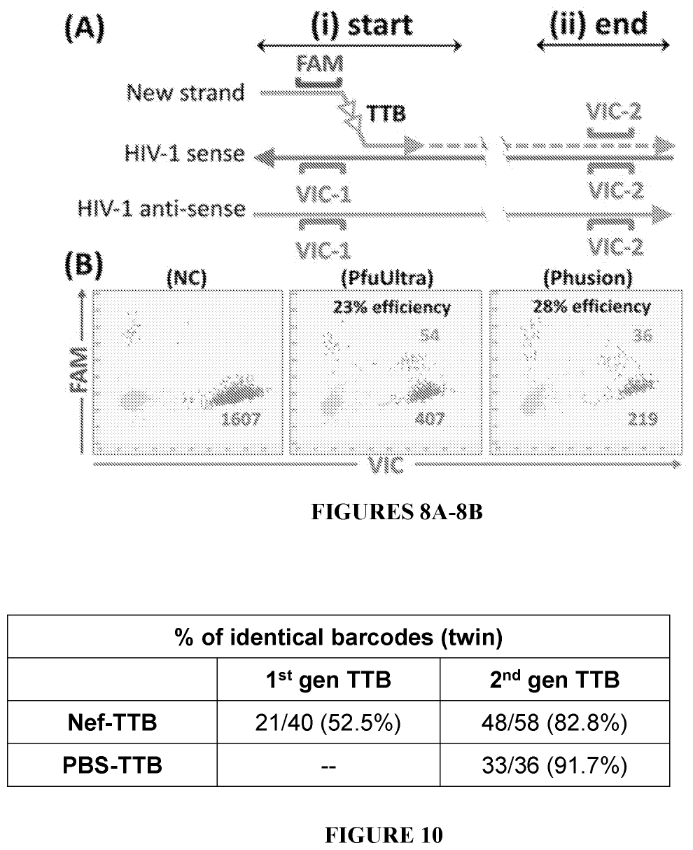

Improving TTB labeling efficiency. Assigning unique TTB to individual target DNA is a key step whose efficiency ultimately determines the sensitivity of the sequencing technology. Long-range primer extension is often inefficient. A preliminary study suggested that multi-stage, multi-enzyme procedures may be required to maximize sensitivity (see FIG. 5). This step consists of two sub-processes: (i) TTB primer binding and initiation of primer extensions and (ii) full-length target extension. The efficiency of these two sub-processes can be quantified by the digital PCR method (FIG. 5) as well as gel electrophoresis (see FIG. 5). (a) Choosing a target-specific primer sequence. Different primer-binding sites and different lengths of target-specific primer sequences using chemically synthesized test TTB-primers are used. Primer-binding efficiency is determined as described above (FIG. 8). (b) Choosing DNA polymerases. (i) TTB-primer binding and initiation of primer extensions and (ii) full-length extension with different DNA polymerases are independently tested. The reaction conditions required for the continuation of these two processes are then optimized. DNA polymerases with a high fidelity and high processivity is tested. (c) Testing substrate DNA conditions. Substrate DNA conditions—for example, chromosomal structure, methylation status, nicking and deletions, the length of DNA, etc., may affect primer binding and full-extension efficiency. Different genomic DNA repair kits, DNA isolation kits, and denaturing conditions can be used to determine the best conditions for the high-efficient TTB labeling.

TTB-mediated, long-range sequencing using HIV-1 laboratory strains. Different DNA and RNA samples are tested, including homogeneous clonal HIV-1 plasmid DNA, proviral DNA from infected cells, and viral RNA genome. The dynamics of viral evolution in the presence of a protease inhibitor, Nelfinavir are also evaluated.

Experimental description: Nanopore sequencing and data analysis. TTB-labeled, PCR-amplified HIV-1 DNA of different lengths—including a 1.5 Kbp gag, a 3 Kbp env, a 6 Kbp gag-pol, and an 8.5 Kbp (near full-length)—are subjected to the standard workflow of the Nanopore 1D2 Sequencing Kit, then run on a MinION device with a R9.5 Flow Cell (or a higher version). Sequence data is filtered and processed for the self-error correction procedures (FIG. 5) using Poretools software and custom-made python scripts. Sequence reads sharing identical TTB undergoes multiple sequence alignments using the Partial-order alignment, the Burrows-Wheeler Aligner, and MegAlign (DNASTAR Inc) to determine a consensus sequence. Evolutionary distance is measured and clustered using the DNADIST program and the “Heatplus” package of the R Bioconductor.

Estimating key experimental parameters using laboratory strains of HIV-1 (NL4.3 and its derivatives). (a) Sequencing a homogeneous DNA pool of HIV-1 plasmid DNA. This analysis assesses (i) the sequencing depths needed to generate a high-accuracy consensus sequence, and (ii) the maximum accuracy achievable with the current read number capacity. To this end, a pool of homogeneous HIV-1 plasmid DNA is prepared from a single bacterial colony, quantified with spectrophotometers and viral DNA-specific digital PCR, and subjected to TTB-mediated, long-range sequencing. Sequencing results are directly compared with the sequence data from Sanger sequencing. Both NL4.3 (WT HIV-1) and NL4.3-EGFP (NL4.3 with an EGFP expression cassette replacing of the env gene) plasmids are used. (b) Serial dilution of a mixed plasmid DNA pool of five known HIV-1 variants. Five different HIV-1 DNA clones with varying genetic mutations as described in previous studies are generated and a mixed pool of known amounts of these clones (plasmids) are prepared for serial dilution studies. Sequencing analysis of these samples allows for the assessment (iii) assay sensitivity, (iv) quantification bias, and (v) reproducibility as well as (vi) PCR recombination types and their rates in sequencing data.

Proviral DNA and RNA analysis. The experimental parameters for infected cell DNA or viral RNA analysis are different than those for sequencing Plasmid DNA. HIV-1 DNA and RNA from human cells is analyzed. (c) Serial dilution of a mixed pool of in vitro infected cells. This experiment helps estimate the key parameters, including sensitivity, quantification bias, reproducibility, and recombination frequency, for proviral DNA samples. Five human cell culture samples (for example, 293T cells)—each infected with five different NL4.3-EGFP variants pseudotyped with vesicular stomatitis virus G-protein (VSVG)—are flow-sorted based on EGFP expression, counted, and pooled at varying ratios (for example, 1:1:2:2:5). The cell pool is serially diluted into background control 293T cells that have been acutely infected with WT NL4.3-EGFP viruses. (d) viral RNA analysis. Viral RNA isolated from viral particles from 293T cells co-transfected with NL4.3-EGFP and VSVG plasmids is reverse-transcribed using a library of (nef-specific) TTB primers. The results of viral cDNA sequencing is compared with those of plasmid sequencing and proviral DNA sequencing.

Drug-resistant HIV-1 development. After establishing the assay parameters using the plasmids and cell controls, in vitro HIV-1 infection model is used to analyze the dynamics of viral evolution in the presence of antiretroviral drugs. A screening assay for HIV-1 drug-resistant variants was previously made using a library of infectious HIV-1 with single nucleotide random mutations in the protease gene. With these established assay conditions, the investigation is: (i) how multiple mutations of protease inhibitor (PI) primary resistance develop in the presence of a PI (nelfinavir) over time and (ii) whether PI-resistant mutation occurs in regions outside the protease gene. Briefly, cells in the PI culture are collected every 3-4 days for 7-10 weeks, and the genomic DNA is subjected to long-range sequencing.

A two-day (48 hour) MinION run generates 100,000-200,000 reads (for the 8.5 Kbp PCR product). Using the error-correction method, the goal is to achieve greater than 99.99% accuracy in the sequencing of the individual HIV-1 genome (less than one error in the whole HIV-1 genome). This can be achieved using a sequencing depth of minimum 20 copies of sequence reads. The TTB approach eliminates the quantification errors associated with uneven PCR amplification, and thereby enables sensitive and accurate detection of low-frequency variants with an efficiency comparable to that of previous studies using NGS.

### Example 7

### Long-Range PCR

Long-range Droplet PCR. A modern means of tackling this challenge is to isolate each DNA molecule prior to PCR, which can be done using droplet PCR. A PCR mixture was partitioned into approximately 20,000 even-sized droplets per PCR tube using a Bio-Rad QX200 Droplet generator (FIG.11A) to perform single DNA amplification in nano-liter (nL) volume droplets. Such single DNA PCR amplification can effectively resolve the PCR recombination issue. Like any other PCR, 9 Kb PCR was challenging with the droplet PCR, but an initial test showed that the efficiency of long-range PCR in droplets can be improved by modification of the PCR reaction premix (FIG. 11B). DNA can be extracted from Droplets by phenol-chloroform extraction.

Single DNA Droplet PCR. When a PCR reaction mixture is partitioned into droplets, some droplets contain one or more copies of the target DNA molecule and some will not contain any targets (Table 4). Droplet PCR is controlled to perform only with 1,000 to 2,000 copies of input target DNA per PCR tube to ensure that the fraction of ≥2 target droplets constitute less than 2.5 to 4.9% of total TTB-DNA. Five to ten PCR tubes of droplet PCR reaction (each containing 1,000 to 2,000 target DNA) generates an ideal range of TTB-DNA appropriate for one MinION sequencing, as the maximum throughput for 9Kb TTB-DNA would be approximately 10,000; see Table 3. The quantity of target DNA can be measured by comparing the frequencies of target-containing droplets and non-target containing droplets [Bio-Rad Droplet Digital PCR (ddPCR) guide] using EvaGreen dye or Taqman Probe (FAM), both of which generate fluorescence in target-containing droplets. Any known fluorescence detection system can be used, including a Bio-Rad QX200 droplet reader, a fluorescence microscope, and a Dual Fluorescence LUNA cell counter (Logos Biosystems). The two-end (duplex) barcoding system can be used to quantify recombination events.

RainDrop digital PCR. Droplet number, size and stability are of key importance in developing long-range droplet PCR. The RainDrop digital PCR system (Bio-Rad), can be used which can generate up to 10 million picoliter-size droplets (500-fold more droplets that are 100-fold smaller than QX200 droplets).

### Example 8

### Improving the Twin Ratios

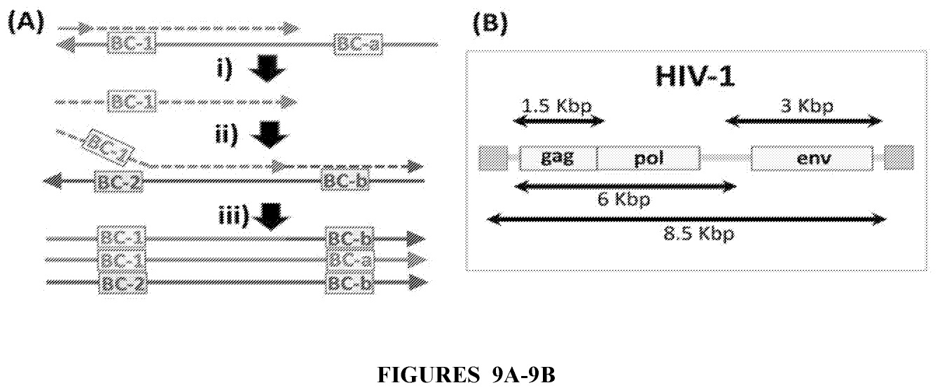

In the first generation TTB library, only half (52.5%) showed two identical barcodes (twins): the remainder showed non-identical two barcodes. The non-identical two-barcodes can be effectively removed by isolating the circularized ssDNA at Steps 1 and 5 (in FIG. 1) by various ways of DNA sizing and isolation methods, including gel (e.g. acrylamide or agarose gel) purification and column chromatography (HPLC or FPLC) methods. After adding these ssDNA isolation in Steps 1 and 5, the twin ratios in the second generation TTB libraries were improved to 82.8% to 91.7%. Nanopore 1D2 sequence analysis for PCR target sequencing has been hampered by the lack of appropriate markers with which to identify the partner sequence. The TTB system can be employed to improve 1D2 sequencing (FIG. 9). To that end, 1D2 adaptor DNA is generated that contains TTB molecules, and they are then used in a library, so that both sense and antisense sequences from the same double stranded DNA have identical tandem twin barcodes (TTBs). TTBs in each adaptor accurately identifies the partners.

### REFERENCES

1. Goodwin, S., McPherson, J. D. & McCombie, W. R. Coming of age: ten years of next-generation sequencing technologies. Nat Rev Genet 17, 333-351 (2016).
2. Fox, E. J., Reid-Bayliss, K. S., Emond, M. J. & Loeb, L. A. Accuracy of Next Generation Sequencing Platforms. Next generation, sequencing & applications 1, 1000106 (2014).
3. Jain, M., Olsen, H. E., Paten, B. & Akeson, M. The Oxford Nanopore MinION: delivery of nanopore sequencing to the genomics community. Genome Biology 17, 239 (2016).
4. Verovskaya, E. et al. Heterogeneity of young and aged murine hematopoietic stem cells revealed by quantitative clonal analysis using cellular barcoding. Blood 122 (2013).
5. Brady, T. et al. A method to sequence and quantify DNA integration for monitoring outcome in gene therapy. Nucleic Acids Research 39, e72 (2011).
6. Gerrits, A. et al. Cellular barcoding tool for clonal analysis in the hematopoietic system. Blood 115, 2610-2618 (2010).
7. Naik, S. H., Schumacher, T. N. & Perié, L. Cellular barcoding: A technical appraisal. Experimental Hematology 42, 598-608 (2014).
8. Jabara, C. B., Jones, C. D., Roach, J., Anderson, J. A. & Swanstrom, R. Accurate sampling and deep sequencing of the HIV-1 protease gene using a Primer ID. Proceedings of the National Academy of Sciences of the United States of America 108, 20166-20171 (2011).
9. Lee, D. F., Lu, J., Chang, S., Loparo, J. J. & Xie, X. S. Mapping DNA polymerase errors by single-molecule sequencing. Nucleic Acids Research 44, el18-e118 (2016).
10. Schmitt, M. W. et al. Sequencing small genomic targets with high efficiency and extreme accuracy. Nat Meth 12, 423-425 (2015).
11. Kinde, I., Wu, J., Papadopoulos, N., Kinzler, K. W. & Vogelstein, B. Detection and quantification of rare mutations with massively parallel sequencing. Proceedings of the National Academy of Sciences 108, 9530-9535 (2011).
12. Hiatt, J. B., Pritchard, C. C., Salipante, S. J., O'Roak, B. J. & Shendure, J. Single molecule molecular inversion probes for targeted, high-accuracy detection of low-frequency variation. Genome Research 23, 843-854 (2013).
13. Schmitt, M. W. et al. Detection of ultra-rare mutations by next-generation sequencing. Proceedings of the National Academy of Sciences 109, 14508-14513 (2012).
14. Bystrykh, L. V. & Belderbos, M. E. in Stem Cell Heterogeneity: Methods and Protocols. (ed. K. Turksen) 57-89 (Springer New York, New York, N.Y.; 2016).
15. Zhou, S., Jones, C., Mieczkowski, P. & Swanstrom, R. Primer ID Validates Template Sampling Depth and Greatly Reduces the Error Rate of Next-Generation Sequencing of HIV-1 Genomic RNA Populations. Journal of Virology 89, 8540-8555 (2015).
16. Boltz, V. F. et al. Ultrasensitive single-genome sequencing: accurate, targeted, next generation sequencing of HIV-1 RNA. Retrovirology 13, 87 (2016).
17. Seifert, D. et al. A Comprehensive Analysis of Primer IDs to Study Heterogeneous HIV-1 Populations. Journal of Molecular Biology 428, 238-250 (2016).
18. Thielecke, L. et al. Limitations and challenges of genetic barcode quantification. 7, 43249 (2017).
19. Brodin, J. et al. Challenges with Using Primer IDs to Improve Accuracy of Next Generation Sequencing. PLoS ONE 10, e0119123 (2015).
20. Morens, D. M. & Fauci, A. S. Emerging Infectious Diseases: Threats to Human Health and Global Stability. PLOS Pathogens 9, e1003467 (2013).
21. Woolhouse, M. E. J., Haydon, D. T. & Antia, R. Emerging pathogens: the epidemiology and evolution of species jumps. Trends in Ecology & Evolution 20, 238-244 (2005).
22. Morens, D. M., Folkers, G. K. & Fauci, A. S. The challenge of emerging and re-emerging infectious diseases. Nature 430, 242-249 (2004).
23. Clavel, F. & Hance, A. J. HIV Drug Resistance. New England Journal of Medicine 350, 1023-1035 (2004).
24. Li, J. Z., Paredes, R., Ribaudo, H. J. & et al. Low-frequency hiv-1 drug resistance mutations and risk of nnrti-based antiretroviral treatment failure: A systematic review and pooled analysis. JAMA 4 305, 1327-1335 (2011).
25. Johnson, J. A. et al. Minority HIV-1 Drug Resistance Mutations Are Present in Antiretroviral Treatment—Naïve Populations and Associate with Reduced Treatment Efficacy. PLOS Medicine 5, e158 (2008).
26. Gianella, S. & Richman, D. D. Minority Variants of Drug-Resistant HIV. The Journal of Infectious Diseases 202, 657-666 (2010).
27. Halvas, E. K. et al. Blinded, Multicenter Comparison of Methods To Detect a Drug-Resistant Mutant of Human Immunodeficiency Virus Type 1 at Low Frequency. Journal of Clinical Microbiology 44, 2612-2614 (2006).
28. Brumme, C. J. & Poon, A. F. Y. Promises and pitfalls of Illumina sequencing for HIV resistance genotyping. Virus Research (2016).
29. Beerenwinkel, N., Günthard, H. F., Roth, V. & Metzner, K. J. Challenges and opportunities in estimating viral genetic diversity from next-generation sequencing data. Frontiers in Microbiology 3, 329 (2012).
30. Bruner, K. M. et al. Defective proviruses rapidly accumulate during acute HIV-1 infection. Nat Med 22, 1043-1049 (2016).
31. Ho, Y.-C. et al. Replication-Competent Noninduced Proviruses in the Latent Reservoir Increase Barrier to HIV-1 Cure. Cell 155, 540-551 (2013).
32. Boritz, Eli A. et al. Multiple Origins of Virus Persistence during Natural Control of HIV Infection. Cell 166, 1004-1015 (2016).
33. Imamichi, H. et al. Defective HIV-1 proviruses produce novel protein-coding RNA species in HIV-infected patients on combination antiretroviral therapy. Proceedings of the National Academy of Sciences 113, 8783-8788 (2016).
34. Yebra, G., Hodcroft, E. B., Ragonnet-Cronin, M. L., Pillay, D. & Brown, A. J. L. Using nearly full-genome HIV sequence data improves phylogeny reconstruction in a simulated epidemic. 6, 39489 (2016).
35. Gibson, R. M. et al. Sensitive Deep-Sequencing-Based HIV-1 Genotyping Assay To Simultaneously Determine Susceptibility to Protease, Reverse Transcriptase, Integrase, and Maturation Inhibitors, as Well as HIV-1 Coreceptor Tropism. Antimicrobial Agents and Chemotherapy 58, 2167-2185 (2014).
36. Dilernia, D. A. et al. Multiplexed highly-accurate DNA sequencing of closely-related HIV-1 variants using continuous long reads from single molecule, real-time sequencing. Nucleic Acids Research 43, e129-e129 (2015).
37. Grossmann, S., Nowak, P. & Neogi, U. Subtype-independent near full-length HIV-1 genome sequencing and assembly to be used in large molecular epidemiological studies and clinical management. Journal of the International AIDS Society 18, 20035 (2015).
38. Smyth, R. P. et al. Identifying Recombination Hot Spots in the HIV-1 Genome. Journal of Virology 88, 2891-2902 (2014).
39. Henn, M. R. et al. Whole Genome Deep Sequencing of HIV-1 Reveals the Impact of Early Minor Variants Upon Immune Recognition During Acute Infection. PLoS Pathogens 8, e1002529 (2012).
40. Cotton, L. A. et al. Genotypic and Functional Impact of HIV-1 Adaptation to Its Host Population during the North American Epidemic. PLoS Genetics 10, e1004295 (2014).
41. Yap, S.-H. et al. N348I in the Connection Domain of HIV-1 Reverse Transcriptase Confers Zidovudine and Nevirapine Resistance. PLoS Medicine 4, e335 (2007).
42. Fun, A., Wensing, A. M. J., Verheyen, J. & Nijhuis, M. Human Immunodeficiency Virus gag and protease: partners in resistance. Retrovirology 9, 63-63 (2012).
43. Dam, E. et al. Gag Mutations Strongly Contribute to HIV-1 Resistance to Protease Inhibitors in Highly Drug-Experienced Patients besides Compensating for Fitness Loss. PLoS Pathogens 5, e1000345 (2009).
44. Levi, J. et al. Can the UNAIDS 90-90-90 target be achieved? A systematic analysis of national HIV treatment cascades. BMJ Global Health 1 (2016).
45. Loman, N. J., Quick, J. & Simpson, J. T. A complete bacterial genome assembled de novo using only nanopore sequencing data. Nat Meth 12, 733-735 (2015).
46. Leggett, R. M., Heavens, D., Caccamo, M., Clark, M. D. & Davey, R. P. NanoOK: multi-reference alignment analysis of nanopore sequencing data, quality and error profiles. Bioinformatics 32, 142-144 (2016).
47. Szalay, T. & Golovchenko, J. A. De novo sequencing and variant calling with nanopores using PoreSeq. Nat Biotech 33, 1087-1091 (2015).
48. Kim, S. et al. Fidelity of Target Site Duplication and Sequence Preference during Integration of Xenotropic Murine Leukemia Virus-Related Virus. PLOS ONE 5, e10255 (2010).
49. Daley, T. & Smith, A. D. Predicting the molecular complexity of sequencing libraries. Nat Meth 10, 325-327 (2013).
50. YANG, Y. L. W., GUANGQIANG; DORMAN, KARIN; and KAPLAN, ANDREW H. Long Polymerase Chain Reaction Amplification of Heterogeneous HIV Type 1 Templates Produces Recombination at a Relatively High Frequency. AIDS Research and Human Retroviruses 12, 303-306 (2009).
51. Shao, W. et al. Analysis of 454 sequencing error rate, error sources, and artifact recombination for detection of Low-frequency drug resistance mutations in HIV-1 DNA. Retrovirology 10, 18-18 (2013).
52. Görzer, I., Guelly, C., Trajanoski, S. & Puchhammer-Stöckl, E. The impact of PCR-generated recombination on diversity estimation of mixed viral populations by deep sequencing. Journal of Virological Methods 169, 248-252 (2010).
53. Judo, M. S., Wedel, A. B. & Wilson, C. Stimulation and suppression of PCR-mediated recombination. Nucleic Acids Research 26, 1819-1825 (1998).
54. Zhang, J.-P. et al. Efficient precise knockin with a double cut HDR donor after CRISPR/Cas9-mediated double-stranded DNA cleavage. Genome Biology 18, 35 (2017).
55. Marx, V. Nanopores: a sequencer in your backpack. Nat Meth 12, 1015-1018 (2015).
56. Lee, C., Grasso, C. & Sharlow, M. F. Multiple sequence alignment using partial order graphs. Bioinformatics 18, 452-464 (2002).
57. Li, H. & Durbin, R. Fast and accurate long-read alignment with Burrows-Wheeler transform. Bioinformatics 26, 589-595 (2010).
58. Zhang, H. et al. Novel Single-Cell-Level Phenotypic Assay for Residual Drug Susceptibility and Reduced Replication Capacity of Drug-Resistant Human Immunodeficiency Virus Type 1. Journal of Virology 78, 1718-1729 (2004).
59. Kim, S. et al. Efficient Identification of Human Immunodeficiency Virus Type 1 Mutants Resistant to a Protease Inhibitor by Using a Random Mutant Library. Antimicrobial Agents and Chemotherapy 55, 5090-5098 (2011).
60. Keys, J. R. et al. Primer ID Informs Next-Generation Sequencing Platforms and Reveals Preexisting Drug Resistance Mutations in the HIV-1 Reverse Transcriptase Coding Domain. AIDS Research and Human Retroviruses 31, 658-668 (2015).
61. Morens, D. M. & Fauci, A. S. Emerging Infectious Diseases: Threats to Human Health and Global Stability. PLOS Pathogens 9, e1003467 (2013).
62. Woolhouse, M. E. J., Haydon, D. T. & Antia, R. Emerging pathogens: the epidemiology and evolution of species jumps. Trends in Ecology & Evolution 20, 238-244 (2005).
63. Morens, D. M., Folkers, G. K. & Fauci, A. S. The challenge of emerging and re-emerging infectious diseases. Nature 430, 242-249 (2004).
64. Bruner, K. M. et al. Defective proviruses rapidly accumulate during acute HIV-1 infection. Nat Med 22, 1043-1049 (2016).
65. Ho, Y.-C. et al. Replication-Competent Noninduced Proviruses in the Latent Reservoir Increase Barrier to HIV-1 Cure. Cell 155, 540-551 (2013).
66. Boritz, Eli A. et al. Multiple Origins of Virus Persistence during Natural Control of HIV Infection. Cell 166, 1004-1015 (2016).
67. Imamichi, H. et al. Defective HIV-1 proviruses produce novel protein-coding RNA species in HIV-infected patients on combination antiretroviral therapy. Proceedings of the National Academy of Sciences 113, 8783-8788 (2016).
68. Yebra, G., Hodcroft, E. B., Ragonnet-Cronin, M. L., Pillay, D. & Brown, A. J. L. Using nearly full-genome HIV sequence data improves phylogeny reconstruction in a simulated epidemic. 6, 39489 (2016).
69. Dilernia, D. A. et al. Multiplexed highly-accurate DNA sequencing of closely-related HIV-1 variants using continuous long reads from single molecule, real-time sequencing. Nucleic Acids Research 43, e129-e129 (2015).
70. Clavel, F. & Hance, A. J. HIV Drug Resistance. New England Journal of Medicine 350, 1023-1035 (2004).
71. Li, J. Z., Paredes, R., Ribaudo, H. J. & et al. Low-frequency hiv-1 drug resistance mutations and risk of nnrti-based antiretroviral treatment failure: A systematic review and pooled analysis. JAMA 305, 1327-1335 (2011).
72. Johnson, J. A. et al. Minority HIV-1 Drug Resistance Mutations Are Present in Antiretroviral Treatment—Naïve Populations and Associate with Reduced Treatment Efficacy. PLOS Medicine 5, e158 (2008).
73. Levi, J. et al. Can the UNAIDS 90-90-90 target be achieved? A systematic analysis of national HIV treatment cascades. BMJ Global Health 1 (2016).
74. Fox, E. J., Reid-Bayliss, K. S., Emond, M. J. & Loeb, L. A. Accuracy of Next Generation Sequencing Platforms. Next generation, sequencing & applications 1, 1000106 (2014).
75. Goodwin, S., McPherson, J. D. & McCombie, W. R. Coming of age: ten years of next-generation sequencing technologies. Nat Rev Genet 17, 333-351 (2016).
76. Schmitt, M. W. et al. Sequencing small genomic targets with high efficiency and extreme accuracy. Nat Meth 12, 423-425 (2015).
77. Verovskaya, E. et al. Heterogeneity of young and aged murine hematopoietic stem cells revealed by quantitative clonal analysis using cellular barcoding. Blood 122 (2013).
78. Brady, T. et al. A method to sequence and quantify DNA integration for monitoring outcome in gene therapy. Nucleic Acids Research 39, e72 (2011).
79. Gerrits, A. et al. Cellular barcoding tool for clonal analysis in the hematopoietic system. Blood 115, 2610-2618 (2010).
80. Naik, S. H., Schumacher, T. N. & Perié, L. Cellular barcoding: A technical appraisal. Experimental Hematology 42, 598-608 (2014).
81. Bystrykh, L. V. & Belderbos, M. E. in Stem Cell Heterogeneity: Methods and Protocols. (ed. K. Turksen) 57-89 (Springer New York, New York, N.Y.; 2016).

1. A library of tandem twin barcode (TTB) oligonucleotide molecules, wherein said library comprises at least 5 unique TTB oligonucleotide molecules, wherein said TTB molecules comprise a first and second barcode sequence, wherein said first and second barcode sequence are identical to each other and positioned in a same 5′ to 3′ orientation, and wherein said TTB oligonucleotide molecules are flanked on either side by two target regions that are common to all TTB oligonucleotides in the library.

2. The library of claim 1, wherein the library comprises at least 10 unique TTB oligonucleotide molecules.

3. The library of claim 2, wherein the library comprises at least 100 unique TTB oligonucleotide molecules.

4. The library of claim 3, wherein the library comprises at least 1000 unique TTB oligonucleotide molecules.

5. The library of claim 4, wherein the library comprises at least 10⁵ unique TTB oligonucleotide molecules.

6. The library of claim 5, wherein the library comprises at least 10⁷ unique TTB oligonucleotide molecules.

7. The library of claim 6 wherein the library comprises at least 10⁹ unique TTB oligonucleotide molecules.

8. The library of claim 7, wherein at least one of the TTB oligonucleotide molecules of the library comprises a spacer between the first and second barcode sequence.

9. The library of claim 8, wherein the spacer is at least 2 nucleotides in length.

10. The library of claim 9, wherein the spacer is at least 5 nucleotides in length.

11. The library of claim 10, wherein the spacer is at least 100 nucleotides in length.

12. The library of claim 1, wherein each of the first and second barcode sequences comprise a barcode block at least 5 nucleotides in length.

13. The library of claim 1, wherein each of the first and second barcode sequences comprise at least one barcode block sequence, wherein said barcode block can be repeated.

14. The library of claim 1, wherein each of said TTB molecules further comprise a target region capable of annealing or ligating to the target nucleic acid.

15. The library of claim 1, wherein the first and second barcode sequence of the TTB molecule uniquely identifies each of the barcode molecules via the barcode block.

16. A method of labeling target polynucleotide molecules with a unique identifier, the method comprising labeling the barcode library of claim 1 with target polynucleotide molecules.

17. The method of claim 16, wherein the target polynucleotide is sequenced after labeling with the barcode library.

18. The method of claim 16, wherein said sequencing can comprise multiplex sequencing, shotgun metagenomic sequencing, targeted sequencing, and droplet (or emersion)-mediated sequencing—using various sequencing platforms, including Sanger-capillary sequencing, Solexa sequencing, Ion Torrent sequencing, SOLiD sequencing, 454 pyrosequencing, Single Molecule Real Time (SMRT) sequencing, and Nanopore Sequencing.

19. A kit for labelling a target nucleic acid for sequencing, wherein the kit comprises a) a library of at least 5 unique TTB molecules, wherein said TTB molecules comprise a first and second barcode sequence, wherein said first and second barcode sequence are identical to each other; and b) reagents for sequencing.

20. The kit of claim 19, wherein said sequencing reagents can comprise various molecular biology reagents, including DNA polymerases, RNA polymerases, Reverse-transcriptases, DNA ligases, RNA ligases, transposases, viral integrase, CRISPR/Cas9, zinc finger nucleases, transcription activator-like effector nucleases, exonucleases, endonucleases, Polynucleotide Kinases, or nucleotides.

21-45. (canceled)
